(19) United States
(12) Patent Application Publication
(10) Pub. No.: US 2017/0037400 A1
(43) Pub. Date: Feb. 9, 2017

(54) MODULATING IMMUNE RESPONSES

(71) Applicant: Glen N. BARBER, Palmetto Bay, FL (US)

(72) Inventor: Glen N. BARBER, Palmetto Bay, FL (US)

(21) Appl. No.: 15/120,694

(22) PCT Filed: Apr. 30, 2013
(86) PCT No.: PCT/US2013/038840

§ 371 Date: Aug. 22, 2016

Related U.S. Application Data
(63) Continuation-in-part of application No. 13/460,408, filed on Apr. 30, 2012.

(51) Int. Cl. C12N 15/113

(52) U.S. Cl. CPC C12N 15/113; C12N 2310/14

(57) ABSTRACT

Modulators of STING are able to upregulate or down regulate immune responses. Administration of such modulators can be used to treat diseases or other undesireable conditions in a subject either directly or in combination with other agents.

### GOVERNMENT RIGHTS

The invention described herein was made with U.S. government support under grant number R01A1079336 awarded by the National Institutes of Health. The U.S. government has certain rights in the invention.

### FIELD OF THE INVENTION

Embodiments of the invention relate to compositions and methods for modulating innate and adaptive immunity in a subject and/or for the treatment of an immune-related disorder, cancer, autoimmunity, treating and preventing infections.

### BACKGROUND

Cellular host defense responses to pathogen invasion principally involves the detection of pathogen associated molecular patterns (PAMPs) such as viral nucleic acid or bacterial cell wall components including lipopolysaccharide or flagellar proteins that results in the induction of anti-pathogen genes. For example, viral RNA can be detected by membrane bound Toll-like receptors (TLR's) present in the endoplasmic reticulum (ER) and/or endosomes (e.g. TLR 3 and 7/8) or by TLR-independent intracellular DExD/H box RNA helicases referred to as retinoic acid inducible gene 1 (RIG-I) or melanoma differentiation associated antigen 5 (MDA5, also referred to as IFIH1 and helicard). These events culminate in the activation of downstream signaling events, much of which remains unknown, leading to the transcription of NF-κB and IRF3/7-dependent genes, including type I IFN.

### SUMMARY

STING (Stimulator of Interferon Genes), a molecule that plays a key role in the innate immune response, includes 5 putative transmembrane (TM) regions, predominantly resides in the endoplasmic reticulum (ER), and is able to activate both NF-κB and IRF3 transcription pathways to induce type I IFN and to exert a potent anti-viral state following expression. See U.S. patent application Ser. No. 13/057,662 and PCT/US2009/052767. Loss of STING reduced the ability of polyIC to activate type I IFN and rendered murine embryonic fibroblasts lacking STING (^{-/-} MEFs) generated by targeted homologous recombination, susceptible to vesicular stomatitis virus (VSV) infection. In the absence of STING, DNA-mediated type I IFN responses were inhibited, indicating that STING may play an important role in recognizing DNA from viruses, bacteria, and other pathogens which can infect cells. Yeast-two hybrid and co-immunoprecipitation studies indicated that STING interacts with RIG-I and with Ssr2/TRAPβ, a member of the translocon-associated protein (TRAP) complex required for protein translocation across the ER membrane following translation. RNAi ablation of TRAPβ inhibited STING function and impeded the production of type I IFN in response to polyIC.

Further experiments showed that STING itself binds nucleic acids including single- and double-stranded DNA such as from pathogens and apoptotic DNA, and plays a central role in regulating proinflammatory gene expression in inflammatory conditions such as DNA-mediated arthritis and cancer. Various new methods of, and compositions for, upregulating STING expression or function are described herein along with further characterization of other cellular molecule which interact with STING. These discoveries allow for the design of new adjuvants, vaccines and therapies to regulate the immune system and other systems.

Described herein are methods for modulating an immune response in a subject having a disease or disorder associated with aberrant STING function. These methods can include the step of administering to the subject an amount of a pharmaceutical composition including an agent which modulates STING function and a pharmaceutically acceptable carrier, wherein amount the pharmaceutical composition is effective to ameliorate the aberrant STING function in the subject. The agent can be a small molecule that increases or decreases STING function, or a nucleic acid molecule that binds to STING under intracellular conditions. The STING-binding nucleic acid molecule can be a single-stranded DNA between 40 and 150 base pairs in length or a double-stranded DNA between 40 and 150, 60 and 120, 80 and 100, or 85 and 95 base pairs in length or longer. The STING-binding nucleic acid molecule can be nuclease-resistant, e.g., made up of nuclease-resistant nucleotides. It can also be associated with a molecule that facilitates transmembrane transport. In these methods, the disease or disorder can be a DNA-dependent inflammatory disease.

Also described herein are methods of treating cancer in a subject having a cancerous tumor infiltrated with inflammatory immune cells. These methods can include the step of administering to the subject an amount of a pharmaceutical composition including an agent which downregulates STING function or expression and a pharmaceutically acceptable carrier, wherein amount the pharmaceutical composition is effective to reduce the number of inflammatory immune cells infiltrating the cancerous tumor by at least 50% (e.g., at least 50, 60, 70, 80, or 90%, or until reduction of inflammatory cell infilitration is detectably reduced by histology or scanning).

### DESCRIPTION OF THE DRAWINGS

FIGS. 1A-G show STING dependent innate immune signaling. FIG. 1A: Human Telomerase Fibroblasts (hTERT-BJ1) were transfected with various nucleotides (3 μg/ml) for 16 h. Endogenous IFNβ levels were measured. hTERT-BJ1 cells were transfected with FITC conjugated dsDNA90 was examined by fluorescent microscopy to ensure efficient transfection. FIG. 1B: hTERT-BJ1 cells were transfected with mock, random or two independent human STING siRNAs (siRNA 3 or 4) for 3 days followed by dsDNA90 transfection (3 μg/ml) for 16 hours. Endogenous IFNβ levels were measured. Silencing of hSTING protein was demonstrated by immunoblotting, with β-actin serving as a control. FIG. 36C: Primary STING^{+/+}, STING^{-/-}, STAT1^{+/+} or STAT1^{-/-} MEFs were transfected with or without dsDNA90 (3 μg/ml) for 3 hours. Total RNA was purified and examined for gene expression by Illumina Sentrix BeadChip Array (Mouse WG6 version 2). FIG. 1D: hTERT-BJ1 cells were treated NS or STING siRNA. After 3 days, cells were treated with dsDNA90, ssDNA90 or ssDNA45 (3 μg/ml). IFNβ mRNA levels were measured by real time RT-PCR after 16 hours. FIG. 1E: hTERT-BJ1 cells were treated NS or STING siRNA. At 3 days, cells were treated with dsDNA90, ssDNA90 or ssDNA45 (3 μg/ml). Endogenous IFNβ levels were measured after 16 hours. FIG. 1F: Primary STING^{+/+} or STING^{-/-} MEFs were transfected with or without dsDNA90 (3 μg/ml). After 3 h, the same as FIG. 1C. FIG. 1G: hTERT-BJ1 cells were treated with or without dsDNA90 (3 μg/ml) for 3 hours and stained with anti-HA antibody and calreticulin as an ER marker.

FIGS. 2A-J show that STING binds to DNA. FIG. 2A: 293T cells were transfected with indicated plasmids. Cell lysates were precipitated with biotin-dsDNA90 agarose and analyzed by immunoblotting using anti-HA antibody. FIG. 2B: Schematic of STING mutants. FIG. 2C: Same as FIG. 2A. FIG. 2D: Same as FIG. 2A. STING variants unable to bind DNA are labeled in red. FIG. 2E: hTERT-BJ1 cells were transfected biotin conjugated dsDNA90 (B-dsDNA90; 3 μg/ml) for 6 h and treated with DSS. Lysates were precipitated using streptavidin agarose and analyzed by immunoblotting using anti-HA antibody. FIG. 2F: STING was expressed in 293T cells and after 36 hours lysates were incubated with dsDNA90 agarose in the presence of competitor dsDNA90, Poly(I:C), B-DNA or ssDNA90 and analyzed by immunoblotting using anti-HA antibody. FIG. 2G: 293T cells were transfected with HA-tagged STING, GFP or TREX1. Cells were lysed and biotin labeled ssDNA or dsDNA added with streptavidin agarose beads. Precipitates were analyzed by immunoblotting using anti-HA antibody. FIG. 2H: 293T cells were transfected with IFNβ-Luciferase and STING variants and luciferase activity measured. FIG. 2I: hTERT-BJ1 cells were transfected with dsDNA90 and crosslinked with formaldehyde. STING was precipitated and bound DNA detected by PCR using dsDNA90 specific primers. NC: negative control. PC: positive control, dsDNA90. FIG. 2J: STING^{+/+} or STING^{-/-} MEFs were transfected with dsDNA90 and then same as FIG. 2I. Error bars indicates s.d. Data are representative of at least two independent experiments.

FIGS. 3A-3H show that TREX1 is negative regulator of STING signaling. FIG. 3A: Immunoblot confirming STING and/or TREX1 knockdown in siRNA treated hTERT-BJ1 cells. FIG. 3B: siRNA treated hTERT-BJ1 cells were transfected with dsDNA90 (3 μg/ml). Endogenous IFNβ levels were measured after 16 hours. *P<0.05. FIG. 3C: siRNA treated hTERT-BJ1 cells were infected with HSV-1 (m.o.i=1) and virus titers measured. *P<0.05. FIG. 38D: siRNA treated hTERT-BJ1 cells were infected with γ34.5 deleted-HSV and virus titers measured. *P<0.05. FIG. 3E: Immunoblot of NS or STING siRNA treated TREX1^{+/+} or TREX1^{-/-} MEFs, confirming STING knockdown. FIG. 3F: siRNA treated TREX1^{+/+} or TREX1^{-/-} MEFs were treated with dsDNA90 and IFNβ levels were measured after 16 hours. *P<0.05. FIG. 3G: siRNA treated TREX1^{+/+} or TREX1^{-/-} MEFs were infected HSV-1 (m.o.i=1) and virus titers measured. *P<0.05. FIG. 3H: Immunofluorescence analysis using anti-TREX1 or anti-STING antibody of hTERT-BJ1 cells transfected with or without dsDNA90. *P<0.05, Student's t-test. Error bars indicated s.d. Data are representative of at least two independent experiments.

FIGS. 4A-J: show TREX1 associates with oligosaccharyltransferase complex. FIG. 4A shows a schematic of TREX1. Red indicates RPN1 binding site. FIG. 4B shows a schematic of STING. Red indicates DAD1 binding site. FIG. 4C: RPN1 interacts with TREX1 in yeast two hybrid analysis (1.pGBKT7, 2.pGBKT7-NFAR M9, 3.pGBKT7-TREX1, 4.pGBKT7-STING full length, 5.pGBKT7-STING C-terminal). FIG. 4D: 293T cells were co-transfected with TREX1-tGFP and RPN1-Myc. Lysates were immunoprecipitated using anti-Myc antibody and analyzed by immunoblotting. FIG. 4H: hTERT-BJ1 cells were treated with or without dsDNA90 (3 μg/ml). At 6 h after transfection, cells were examined by immunofluorescence using anti-STING or anti-DAD1 antibody. FIG. 4I: Immunoblot analysis of microsome fractions after sucrose gradient centrifugation using indicated antibodies. I: input. FIG. 4J: hTERT-BJ1 cells were treated with TREX1, Sec61A1, TRAPβ, NS or STING siRNA. After 72 h, cells were treated with dsDNA90 (3 μg/ml) for 16 h and then endogenous IFNβ levels were measured. *P<0.05, Student's t-test. Error bars indicated s.d. Data are representative of at least two independent experiments.

FIGS. 5A-G show that cytoplasmic DNA induces STING-dependent genes in hTERT-BJ1 cells. FIG. 5A: hTERT-BJ1 cells were treated with STING siRNA. After 3 days, cells were treated with or without dsDNA90 for 3 h. Total RNA was purified and examined for gene expression using Human HT-12_V4_Bead Chip. FIGS. 5B-G: hTERT-BJ 1 cells were treated as in FIG. 5A. Total RNAs were examined by real time PCR for IFNβ (FIG. 5B), PMAIP1 (FIG. 5C), IFIT1 (FIG. 5D), IFIT2 (FIG. 5E), IFIT3 (FIG. 5F) and PTGER4 (FIG. 5G). Real time PCR was performed using TaqMan gene Expression Assay (Applied Biosystem). *P<0.05, Student s t-test. Error Bars indicated s.d. Data are representative of at least two independent experiments.

FIGS. 6A-H show that STING-dependent genes are induced by cytoplasmic DNA in MEFs. Primary STAT1^{+/+} or STAT1^{-/-} MEFs were treated dsDNA90, IFNα or dsDNA90 with IFNα. Total RNAs were purified and examined by real time PCR for IFNβ (FIG. 6A), IFIT1 (FIG. 6B), IFIT2 (FIG. 6C), IFIT3 (FIG. 6D), CXCL 10 (FIG. 6E), GBP1 (FIG. 6F), RSAD2 (FIG. 6G) and CCL5 (FIG. 6H). *P<0.05, Student's t-test. Error Bars indicated s.d. Data are representative of at least two independent experiments.

FIGS. 7A-H show that cytoplasmic DNAs induce STING-dependent genes in MEFs. In FIGS. 7A-H, STING^{+/+} or STING^{-/-} MEFs were treated with or without dsDNA45, dsDNA90, ssDNA45 or ssDNA90 for 3 h. Total RNAs were purified and examined by real time PCR for IFNβ (FIG. 7A), IFIT1 (FIG. 7B), IFIT2 (FIG. 7C), IFIT3 (FIG. 7D), CCL5 (FIG. 7E), CXCL 10 (FIG. 7F), RSAD2 (FIG. 7G) or GBP1 (FIG. 7H). *P<0.05, Student's t-test. Error Bars indicated s.d. Data are representative of at least two independent experiments.

FIGS. 8A-D show STING localization and dimerization. FIG. 8A: MEFs stably expressing STING-HA were treated with ssDNA45, dsDNA45, ssDNA90 or dsDNA90. After 3 h, cells were stained using anti-HA or anti-calreticulin antibody. FIG. 8B: 293T cells were transfected with STING-HA and Myc-STING. Lysates were precipitated by anti-Myc antibody and analyzed by immunoblotting using anti-HA antibody. FIG. 8C. hTERT-BJ 1 cells were treated with or without the cross linker DSS. Cell lysates were subjected to immunoblot using anti-STING antibody. FIG. 8D: 293T cells were transfected with indicated plasmids and treated with DSS. Cell lysates were analyzed by immunoblotting using anti-HA antibody.

FIGS. 9A-I show that DNA virus induces STING-dependent genes in MEFs. FIG. 9A: MEFs were infected with γ34.5 deleted-HSV (m.o.i.=1) for 3 h. Total RNA was purified and examined for gene expression using Illumina Sentric Bead Chip array (Mouse WGS version2). FIGS. 9B-I: STING^{+/+}, STING^{-/-} or STAT^{-/-}/STING^{+/+} MEFs were treated with or without dsDNA90, HSV or γ34.5 deleted-HSV for 3 h. Total RNAs were purified and examined by real time PCR for IFNβ (FIG. 9B), IFIT1 (FIG. 9C), IFIT2 (FIG. 9D), IFIT3 (FIG. 9E), CCL5 (FIG. 9F), CXCL 10 (FIG. 9G), RSAD2 (FIG. 9H) or GBP1 (FIG. 9I). Error Bars indicate s.d.

FIGS. 10A-F show that STING interacts with IRF3/7 and NFκ-B. FIG. 10A: IRF7 binding sites in the promoter regions of STING dependent dsDNA90 stimulatory genes. FIGS. 10B-D: Nuclear extract was isolated from mock treated or dsDNA90 treated STING^{+/+} or STING^{-/-} MEFs and were examined for IRF3 (FIG. 10B), IRF7 (FIG. 10C) and NF-κB (FIG. 10D) activation following the manufacturer's instruction. Nuclear extract kit, TransAM IRF3, TransAM IRF7 and TransAMNFκB family Elisa kits were from Active Motif. *P<0.05, Student s t-test. Error Bars indicated s.d. Data are representative of at least two independent experiments. FIG. 10E: STING^{+/+} or STING^{-/-} MEFs were treated with poly(I:C). dsDNA90 or HSV1 and cells were stained by anti-IRF3 antibody. FIG. 10F. STING^{+/+} or STING^{-/-} MEFs were treated dsDNA90 or HSV1 and cells were stained by anti-p65 antibody. Loss of STING did not affect poly(I:C) mediated innate immune signaling.

FIGS. 11A-F show that STING binds DNA in vitro. FIG. 11A: In vitro translation products were precipitated with biotin conjugated dsDNA90 and immunoblotted by anti-HA antibody. FIG. 11B: schematic of STING variants. FIGS. 11C, 11D: Same as FIG. 11A. STING variants lacking aa 242-341 (red) failed to bind DNA. FIGS. 11E-F: In vitro translation products were precipitated with biotin conjugated ssDNA90 and immunoblotted by anti-HA antibody.

FIGS. 12A-G show that STING binds DNA in vivo and in vitro. hTERT BJ1 cells were transfected with biotin-dsDNA90 and crosslinked by UV. Cells were lysed and precipitated by streptavidin agarose and analyzed by immunoblotting. FIGS. 12B-C hTERT-BJ1 cells were treated with IFI16 (FIG. 12B) or STING (FIG. 12C) siRNA and then same as in FIG. 12A. FIG. 12D: STING^{+/+} or STING^{-/-} MEFs were treated as in FIG. 12A. FIG. 12E: STING-Flag expressing 293T cells were treated with or without biotin-dsDNA90 and crosslinked by DSS. Lysates were precipitated and analyzed by immunoblotting. FIG. 12F: 293T cells were transfected with dsDNA90 or ssDNA90 and crosslinked by UV or DSS and then precipitated and analyzed by immunoblotting. FIG. 12G: STING-Flag expression 293T cells were lysed and incubated with ds0NA90 or Poly(I:C) and biotin-dsDNA90 agarose and then same as FIG. 12E.

FIGS. 13A-C show that STING binds viral DNA. FIG. 13A: Oligonucleotide sequences of HSV, cytomegalovirus (CMV) or adenovirus (ADV). FIGS. 13B-C: 293T cells were transfected with indicated plasmids. Cell-lysates were precipitated with biotin-dsDNA90, biotin-HSV DNA 120 mer, biotin-ADV DNA 120 mer or biotin-CMV DNA 120 mer agarose and analyzed by immunoblotting using anti-HA antibody.

FIGS. 14A-C show that STING binds DNA. FIG. 14A: Schematic of STING ELISA. FIG. 14B: Process of an embodiment of a STING ELISA. FIG. 14C: Binding capacity of dsDNA90 was measured by ELISA. *P<0.05, Student s t-test. Error Bars indicated s.d. Data are representative of at least two independent experiments.

FIGS. 15A-D show that TREX1 is a negative regulator of STING signalling. FIG. 15A: hTERT-BJ1 cells were treated with TREX1, STING or STING and TREX1 siRNA. After 3 days, cells were infected with HSV-Luc (m.o.i=1) for 48 h. Lysates were measured luciferase activity. FIG. 15B: Primary TREX1^{+/+} or TREX^{-/-} MEFs were transfected with NS or STING siRNA. After 3 days, cells were infected with HSV-Luc (m.o.i.=1) for 48 h. Lysates were measured. FIG. 15C: Primary TREX1^{+/+} or TREX1^{-/-} MEFs were transfected with wild type TREX1. After 48 h, cells were infected with HSV and measured HSV replication. TREX1 expression was checked by immunoblotting. *P<0.05, Student's t-test. Error Bars indicated s.d. Data are representative of at least two independent experiments. FIG. 15D: STING^{+/+} or STING^{-/-} MEFs were treated with or without dsDNA90 for 3 h. Total RNAs were purified and examined for gene expression by Illumina Sentrix Bead Chip array (Mouse WG6 version2).

FIG. 16 shows that STING regulates ssDNA90-mediated IFNβ production in TREX^{-/-} MEFs. siRNA treated TREX1^{+/+} or TREX1^{-/-} MEFs were treated with ssDNA45 or ssDNA90 and IFNβ levels were measured after 16 hours. *P<0.05, Student's t-test. Error Bars indicated s.d. Data are representative of at least two independent experiments.

FIGS. 17A-H show that TREX1 is not a negative regulator of STING-dependent genes. FIG. 17A: TREX1^{+/+} or TREX1^{-/-} MEFs were treated with HSV1, IFNα, dsDNA90, triphosphate RNA (TPRNA) and VSV. TPRNA and VSV weakly activated IFN induced genes. Total RNAs were purified and examined for gene expression by Illumina Sentrix Bead Chip array (Mouse WG6 version2). FIGS. 17B-H: Total RNAs were examined by RT-PCR for IFNβ (FIG. 17B), IFIT1 (FIG. 17C), IFIT2 (FIG. 17D), IFIT3 (FIG. 17E), CCL5 (FIG. 17F), CXCL 10 (FIG. 17G), RSAD2 (FIG. 17H). No significant difference in IFN induced genes was observed in TREX1 lacking cells. *P<0.05, Student's t-test. Error bars indicated s.d. Data are representative of at least two independent experiments.

FIGS. 18A-D show that TREX1 associates with oligosaccharyltransferase complex. An IFN-treated hTERT cDNA library was used to develop a yeast two hybrid library (AH109). Full length TREX 1 was used as bait to screen the library. Approximately 5 million cDNA expressing yeast were screened (Clontech). 44 clones were isolated from 3 independent yeast mating procedures. RPN1 was isolated 8 times in total (three times in screen 1, twice in screen 2 and three times in screen 3). The majority of the clones, aside from RPN1, failed to interact with IREX1 after re-testing. Of these eight RPN1 isolated clones, four clones encoded aa 258-397, two clones aa 220-390 and two clones aa 240-367. TREX1 variants were generated and the interation between TREX1 (aa. 241-369) and RPN 1 (aa 256-397) was mapped. To isolate DAD1, the C-terminal region of STING (aa 173-379) was used to screen the same library. 24 isolated clones, full length DAD I was found twice. The majority of the clones, aside from DAD1, failed to interact with STING after re-confirmation studies. Full length DAD1 was seen to associate with region 242-310 of STING. FIG. 18A: Schematic of TREX1 mutants. FIG. 18B: GAL4 binding domain fused to TREX1 or TREX1-4 interact with RPN1 fused to the GAL4 activation domain in yeast two hybrid screening. FIG. 18C: Schematic of STING mutants. FIG. 18D. GAL4 binding domain fused to STING-C-terminal or STING-C2 interact with DAD1 fused to the GAL4 activation domain in yeast two hybrid screening.

FIGS. 19A-C show that TREX1 and STING associate with oligosaccharyltransferase complex. FIG. 19A: 293T cells were co-transfected with TREX1-tGFP and RPN1-Myc. Lysates were immunoprecipitated with anti-tGFP antibody or IgG control and analyzed by immunoblotting using indicated antibodies. FIG. 19B: 293T cells were co-transfected with Myc-STING or GFP-DAD1 and cells were lysed. Lysates were immunoprecipitated with anti-Myc antibody or IgG control and immunoblotted using anti-GFP antibody. FIG. 19C: 293T cells were co-transfected with TREX1-tGFP and RPN1-Myc, GFP-DAD1 or STING-HA. Lysates were immunoprecipitated by anti-tGFP antibody or IgG control and analyzed by immunoblotting using anti-tGFP, anti-Myc, anti-GFP or anti•HA antibodies.

FIG. 20 shows that TREX1 localizes in the endoplasmic reticulum. hTERT-BJ1 cells were transfected with RPN1-Myc. After 48 h, cells were examined by immunofluorescence using anti-TREX1 antibody (red), anti-Myc antibody (green) or anti-calreticulin antibody (blue) as an endoplasmic reticulum marker.

FIGS. 21A-H show that exogenously expressed STING in 293T cells reconstitutes dsDNA90 response. FIG. 21A: 293T cells were infected with control lentivirus or hSTING lentivirus. 1 day after infection, cells were treated with dsDNA90. After 6 h, cells were stained using anti-STING or anti-calreticulin antibody. FIG. 21B: Cell lysates (from FIG. 56A) were subjected to immunoblot using anti-STING antibody. FIGS. 21C-D: Lentivirus infected 293T cells were stimulated with dsDNA90 for 6 h. Total RNAs were purified and examined by real time PCR for IFNβ (FIG. 21C) or IFIT2 (FIG. 21D). FIG. 21E: 293T cells stably transduced with control or hSTING lentivirus were subjected to Brefeldin A (BFA) experiment as shown in the flow chart. FIG. 21F: Cell lysates (from FIG. 21E) were subjected to immunoblot using anti-STING antibody. FIG. 21G: Cell lysates (from FIG. 21E) were measured for IFNβ-luciferase activity. FIG. 21H: Primary STING^{-/-}.MEFs were stably transduced with control or mSTING. Cells were treated with dsDNA90 and endogenous IFNβ levels were measured by ELISA. *P<0.05, Student s t-test. Error bars indicated s.d. Data are representative of at least two independent experiments.

FIG. 22 shows that translocon members regulate HSV1 replication. hTERT-BJ1 cells were treated with TREX1, Sec61A1, TRAPβ, NS or STING siRNA.

FIGS. 23A-C show that IFI16 is not required for IFN-production by dsDNA90 in hTER-BJ1 cells. FIG. 23A: hTERT-B. It cells were treated with NS, IFI16, STING or TREX1 siRNA. After 3 days, cells were lysed and checked expression levels by immunoblotting. FIG. 23B: siRNA treated hTERT-BJ1 cells were stimulated with dsDNA90 and IFNβ production was measured by ELISA. FIG. 23C: siRNA treated hTERT-BJ1 cells were infected with HSV-luciferase (m.o.i=0.1). At 2 days after infection, cells were lysed and luciferase activity was measured. *P<0.05, Student's t-test. Error bars indicated s.d. Data are representative of at least two independent experiments.

FIG. 24 shows an embodiment of a STING cell based assay.

FIG. 25 shows that Drug “A” induces STING trafficking.

FIG. 26 shows that drug “X” inhibits IFNβ mRNA production.

FIG. 27 is a schematic showing that STING is phosphorylated in response to cytoplamic DNA. hTERT-BJ1 cells were transfected with 4 μg/ml of ISD for 6 h. The cell lysates were prepared in TNE buffer and then subjected to immunoprecipitation with anti-STING antibody followed by SDS-PAGE. The gel was stained with CBB and then bands including STING were analyzed by mLC/MS/MS at the Harvard Mass Spectrometry and Proteomics Resource Laboratory. Alignment of STING amino acid sequence from different species and the phosphorylation sites identified by mass spectrometry. Serine 345, 358, 366, and 379 were identified by mass spectrometry. Serine 358 and S366 are important for STING function.

FIG. 28 shows that Serine 366 (S366) of STING is important for IFNβ production in cytoplasmic DNA pathway. 293T cells were transfected with plasmid encoding mutant STING and reporter plasmid. After 36 hr, luciferase activity was measured. STING^{-/-} MEF cells were reconstituted with mutant STING and then the amount of IFNβ in culture media was measured by ELISA. S366 is important for IFN production by STING and S358 is also likely to play an important role.

FIGS. 29A-D show that STING deficient mice are resistant to DMBA induced inflammation and skin oncogenesis: STING^{+/+} and STING^{-/-} mice were either mocked treated with acetone or treated with 10 μg of DMBA on the shaved dorsal weekly for 20 weeks. FIG. 29A: STING deficient animals are resistant to DNA-damaging agents that cause skin cancer. Percentages of skin tumor-free mice were shown in the Kaplan-Meier curve. FIG. 29B: Pictures of representative mice of each treatment groups were shown. FIG. 29C: Histopathological examinations were performed by H&E staining on mock or DMBA treated skin/skin tumor biopsies. Images were taken at 20× magnification. FIG. 29D: Cytokine upregulation in STING expressing mice exposed to carcinogens. RNAs extracted from mock or DMBA treated skin/skin tumor biopsies were analyzed by Illumina Sentrix BeadChip Array (Mouse WG6 version 2) in duplicate. Total gene expression was analyzed. Most variable genes were selected. Rows represent individual genes; columns represent individual samples. Pseudo-colors indicate transcript levels below, equal to, or above the mean (green, black, and red, respectively). Gene expression; fold change log 10 scale ranges between −5 to 5. No cytokines were observed in the skin of STING-deficient animals.

### DETAILED DESCRIPTION

Described herein are methods and compositions for modulating an immune response in a subject having a disease or disorder associated with aberrant STING function. The below described preferred embodiments illustrate adaptation of these compositions and methods. Nonetheless, from the description of these embodiments, other aspects of the invention can be made and/or practiced based on the description provided below.

Methods and compositions for modulating an immune response in a subject (e.g., a human being, dog, cat, horse, cow, goat, pig, etc.) having a disease or disorder associated with aberrant STING function involve a pharmaceutical composition including an agent which modulates STING function and a pharmaceutically acceptable carrier, wherein amount the pharmaceutical composition is effective to ameliorate the aberrant STING function in the subject.

Diseases or disorders associated with aberrant STING function can be any where cells having defective STING function or expression cause or exacerbate the physical symptoms of the disease or disorder. Commonly, such diseases or disorders are mediated by immune system cells, e.g., an inflammatory condition, an autoimmune condition, cancer (e.g., breast, colorectal, prostate, ovarian, leukemia, lung, endometrial, or liver cancer), atherosclerosis, arthritis (e.g., osteoarthritis or rheumatoid arthritis), an inflammatory bowel disease (e.g., ulcerative colitis or Crohn's disease), a peripheral vascular disease, a cerebral vascular accident (stroke), one where chronic inflammation is present, one characterized by lesions having inflammatory cell infiltration, one where amyloid plaques are present in the brain (e.g., Alzheimer's disease), Aicardi-Goutieres syndrome, juvenile arthritis, osteoporosis, amyotrophic lateral sclerosis, or multiple sclerosis.

The agent can be a small molecule (i.e., an organic or inorganic molecule having a molecular weight less than 500, 1000, or 2000 daltons) that increases or decreases STING function or expression or a nucleic acid molecule that binds to STING under intracellular conditions (i.e., under conditions inside a cell where STING is normally located). The agent can also be a STING-binding nucleic acid molecule which can be a single-stranded (ss) or double-stranded (ds) RNA or DNA. Preferably the nucleic acid is between 40 and 150, 60 and 120, 80 and 100, or 85 and 95 base pairs in length or longer. The STING-binding nucleic acid molecule can be nuclease-resistant, e.g., made up of nuclease-resistant nucleotides or in cyclic dinucleotide form. It can also be associated with a molecule that facilitates transmembrane transport.

Methods and compositions for treating cancer in a subject having a cancerous tumor infiltrated with inflammatory immune cells involve a pharmaceutical composition including an agent which downregulates STING function or expression and a pharmaceutically acceptable carrier, wherein amount the pharmaceutical composition is effective to reduce the number of inflammatory immune cells infiltrating the cancerous tumor by at least 50% (e.g., at least 50, 60, 70, 80, or 90%, or until reduction of inflammatory cell infiltration is detectably reduced by histology or scanning).

The compositions described herein might be included along with one or more pharmaceutically acceptable carriers or excipients to make pharmaceutical compositions which can be administered by a variety of routes including oral, rectal, vaginal, topical, transdermal, subcutaneous, intravenous, intramuscular, insufflation, intrathecal, and intranasal administration. Suitable formulations for use in the present invention are found in Remington's Pharmaceutical Sciences, Mack Publishing Company, Philadelphia, Pa., 17th ed. (1985).

The active ingredient(s) can be mixed with an excipient, diluted by an excipient, and/or enclosed within a carrier which can be in the form of a capsule, sachet, paper or other container. When the excipient serves as a diluent, it can be a solid, semi-solid, or liquid material, which acts as a vehicle, carrier or medium for the active ingredient. The compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, sterile liquids for intranasal administration (e.g., a spraying device), or sterile packaged powders. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

For preparing solid formulations such as tablets, the composition can be mixed with a pharmaceutical excipient to form a solid preformulation composition containing a homogeneous mixture of a compound. Tablets or pills may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid forms of the formulations include suspensions and emulsions. To enhance serum half-life, the formulations may be encapsulated, introduced into the lumen of liposomes, prepared as a colloid, or incorporated in the layers of liposomes. A variety of methods are available for preparing liposomes, as described in, e.g., Szoka, et al., U.S. Pat. Nos. 4,235,871, 4,501,728 and 4,837,028 each of which is incorporated herein by reference.

The compositions are preferably formulated in a unit dosage form of the active ingredient(s). The amount administered to the patient will vary depending upon what is being administered, the purpose of the administration, such as prophylaxis or therapy, the state of the patient, the manner of administration, and the like all of which are within the skill of qualified physicians and pharmacists. In therapeutic applications, compositions are administered to a patient already suffering from a disease in an amount sufficient to cure or at least partially arrest the symptoms of the disease and its complications. Amounts effective for this use will depend on the disease condition being treated as well as by the judgment of the attending clinician depending upon factors such as the severity of the symptoms, the age, weight and general condition of the patient, and the like.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

### Example 1

### Translocon-Associated STING Complexes with Cytoplasmic DNA to Regulate Innate Immunity

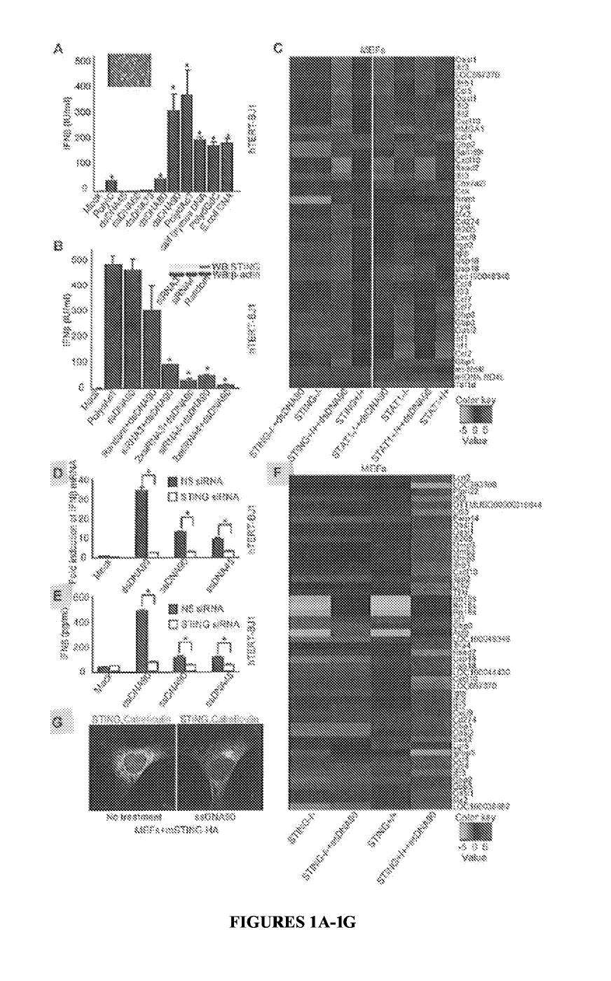

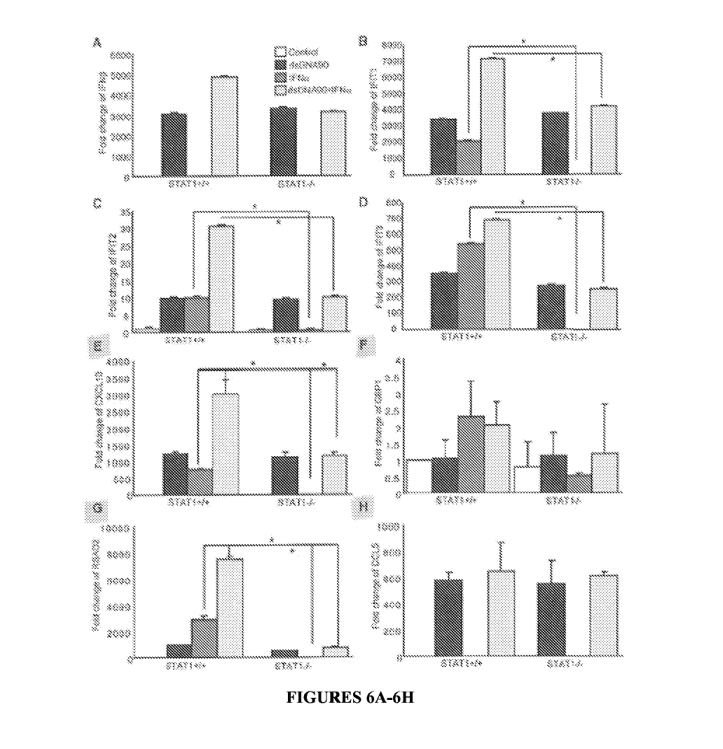

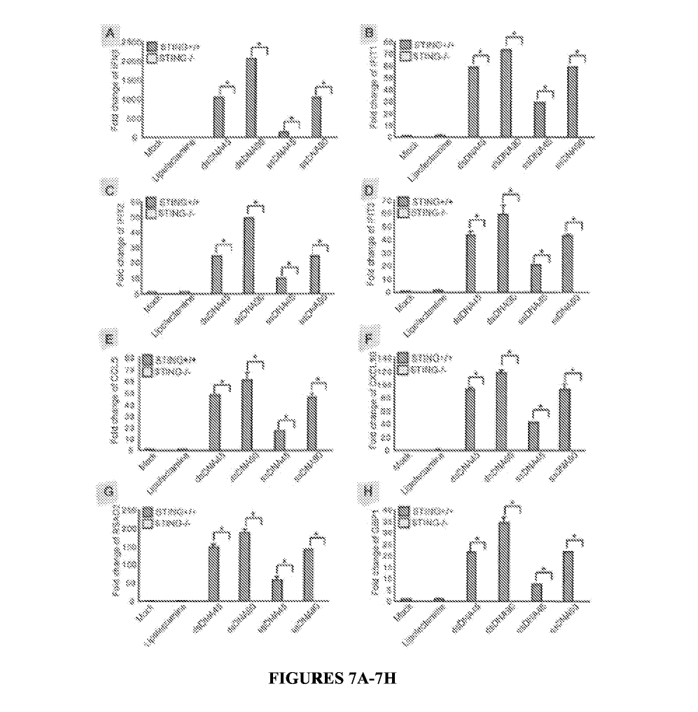

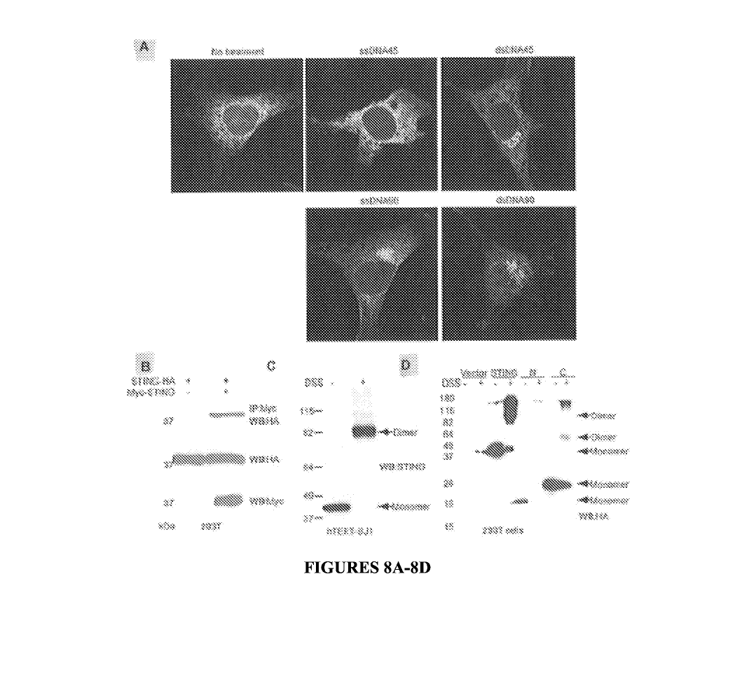

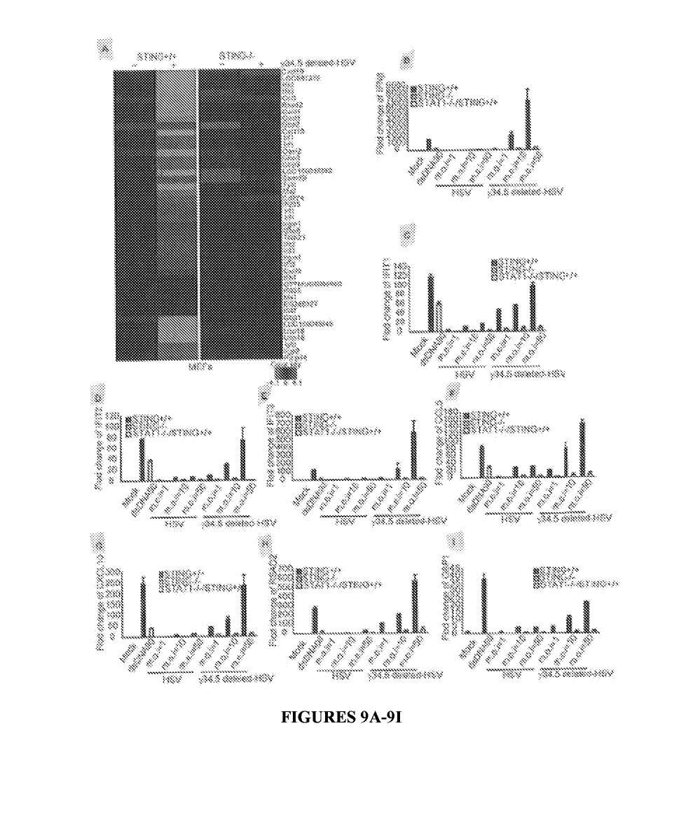

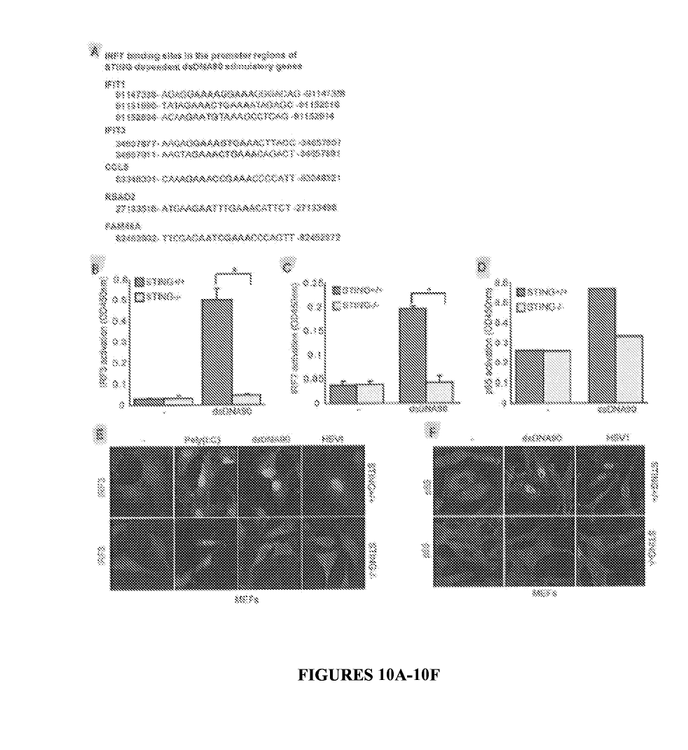

Previously the isolation of a new transmembrane component of the endoplasmic reticulum (ER), referred to as STING (Stimulator of Interferon Genes), which was demonstrated as essential for the production of type I IFN in fibroblasts, macrophages and dendritic cells (DC's) in response to cytoplasmic dsDNA as well as DNA viruses and intracellular bacteria was described (see U.S. patent application Ser. No. 13/057,662 and PCT/US2009/052767). The minimum size of dsDNA required to activate STING-dependent type I IFN signaling in murine cells was noted to be approximately 45 base pairs in murine cells. In normal human cells (hTERT), however, it was observed that dsDNA of approximately 90 base pairs (referred to herein as interferon stimulatory dsDNA90) were required to fully activate type I IFN. Using RNAi knockdown procedures, it was additionally confirmed that STING is indeed essential for the production of type I IFN in hTERTs (FIG. 1B). Further analysis using microarray procedures to measure mRNA expression indicated that cytoplasmic dsDNA can induce a wide array of innate immune genes, in addition to type I IFN, in hTERT cells (FIGS. 5A-G). The induction of these innate molecules which included members of the IFIT family appeared STING-dependent since RNAi knockdown of STING in hTERTs greatly eliminated their stimulation by cytoplasmic dsDNA (FIGS. 5A-G). That cytoplasmic dsDNA induced a variety of innate immune genes in a STING-dependent manner was confirmed using STING^{+/+} or ^{-/-} murine embryonic fibroblasts (MEFs) (FIG. 1C). To confirm that the induction of these mRNAs were STING-dependent genes (SDG) and not stimulated through type I IFN dependent autocrine or paracrine signaling, type I IFN-signaling defective STAT1^{-/-} MEF's were similarly treated with dsDNA and verified that the production of the SDG's remained unaffected (FIG. 1C). Reverse-transcriptase (RT) PCR analysis confirmed the array results (FIGS. 6A-H and 7A-H). It was noted that ssDNA of 45 nucleotides (ssDNA45) weakly induced innate immune gene production in hTERTS and less so in MEFs. However, transfected ssDNA comprising 90 nucleotides (ssDNA90) was observed to more robustly activate an array of genes, including type I IFN in hTERT cells (FIGS. 1D, 1E). That cytoplasmic ssDNA90 induced the production of innate immune genes in a STING-dependent manner was similarly confirmed using STING^{+/+} or ^{-/-} murine embryonic fibroblasts (MEFs) (FIGS. 1F and 7A-H). It was observed that STING likely resides as a homodimer in the ER of both human and murine cells, and migrates from the ER to perinuclear regions in the presence of cytoplasmic ssDNA or dsDNA ligands to activate type I IFN-dependent transcription factors (FIGS. 1G and 8A-E). HSV1 was similarly observed to activate innate immune gene production in a STING dependent manner (FIGS. 9A-I). It was confirmed that many of the SDG's contained IRF7 binding sites in their promoter region (FIGS. 10A-F). Thus, cytoplasmic ssDNA or dsDNA, which includes transfected plasmid DNA, can potently induce the transcription of a wide array of innate immune related genes that is dependent on STING.

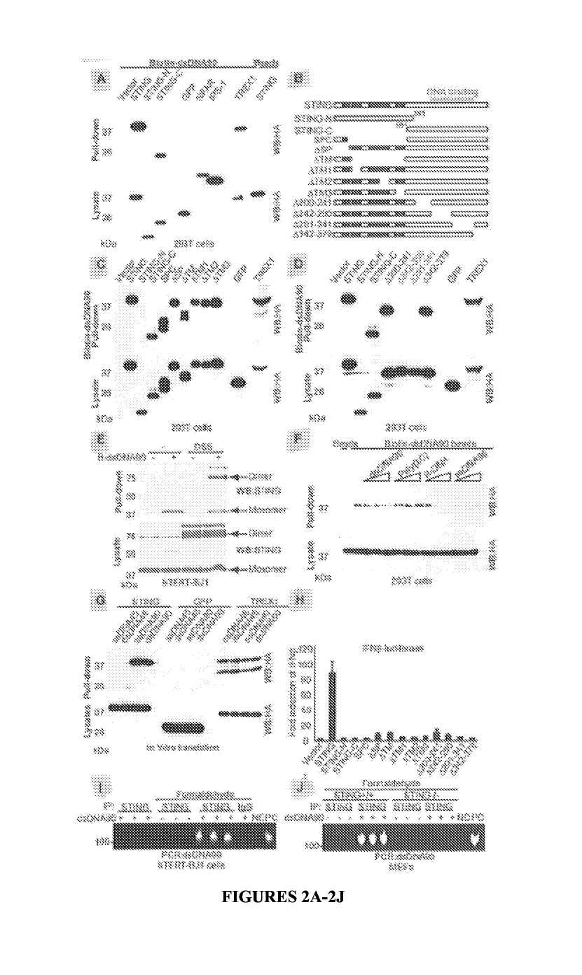

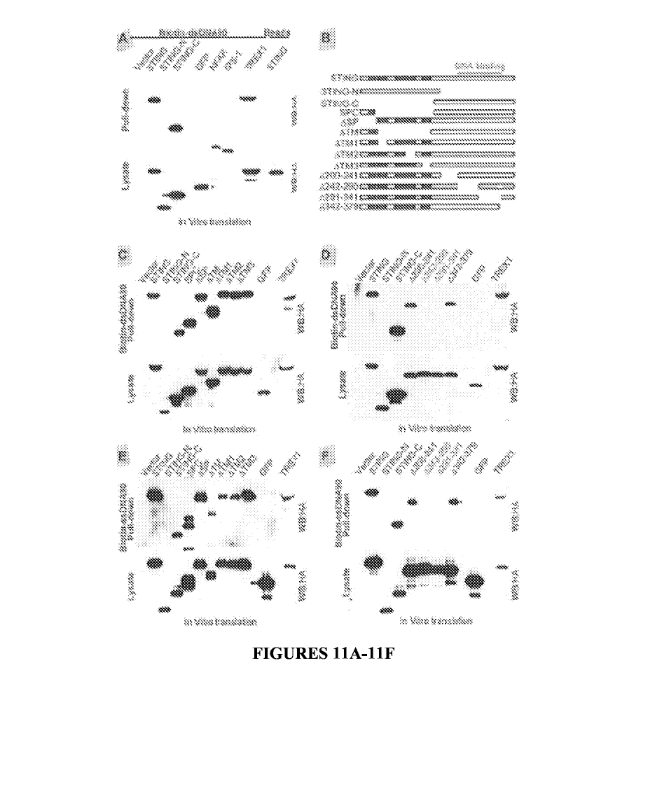

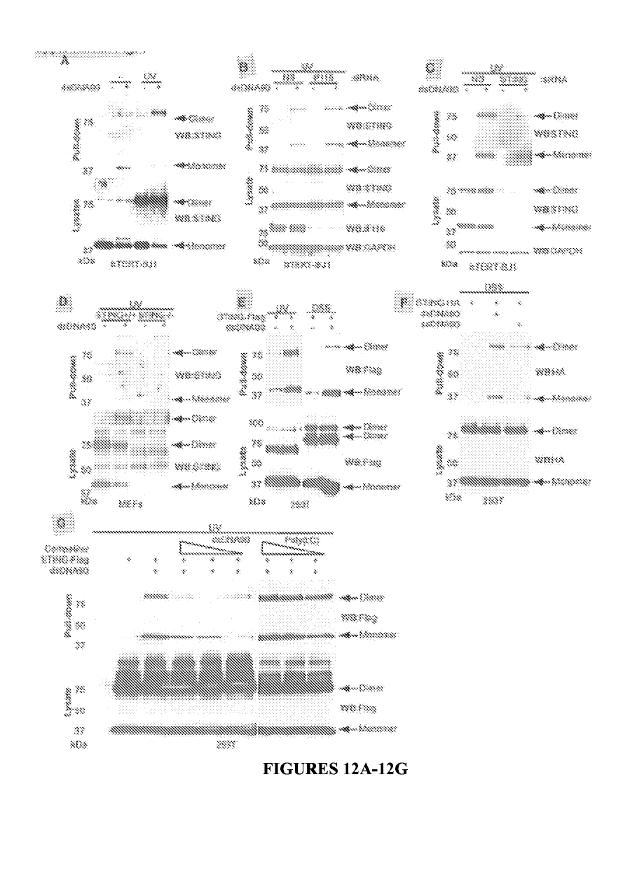

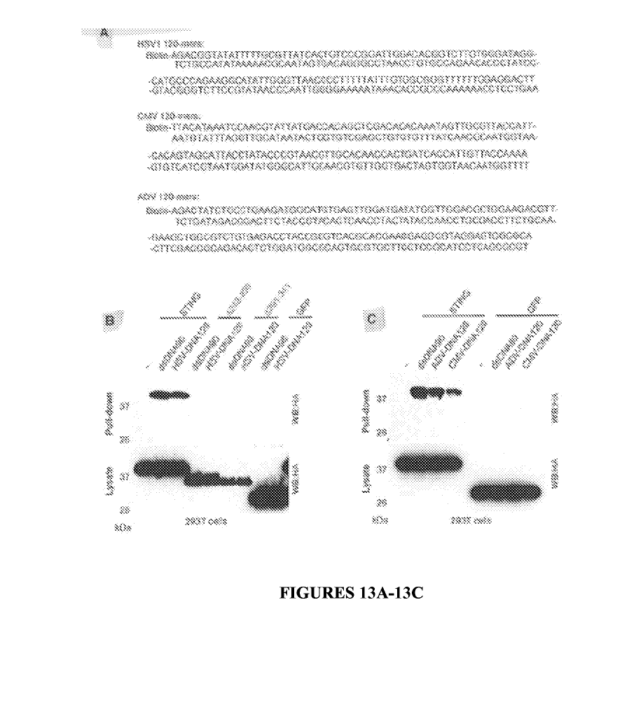

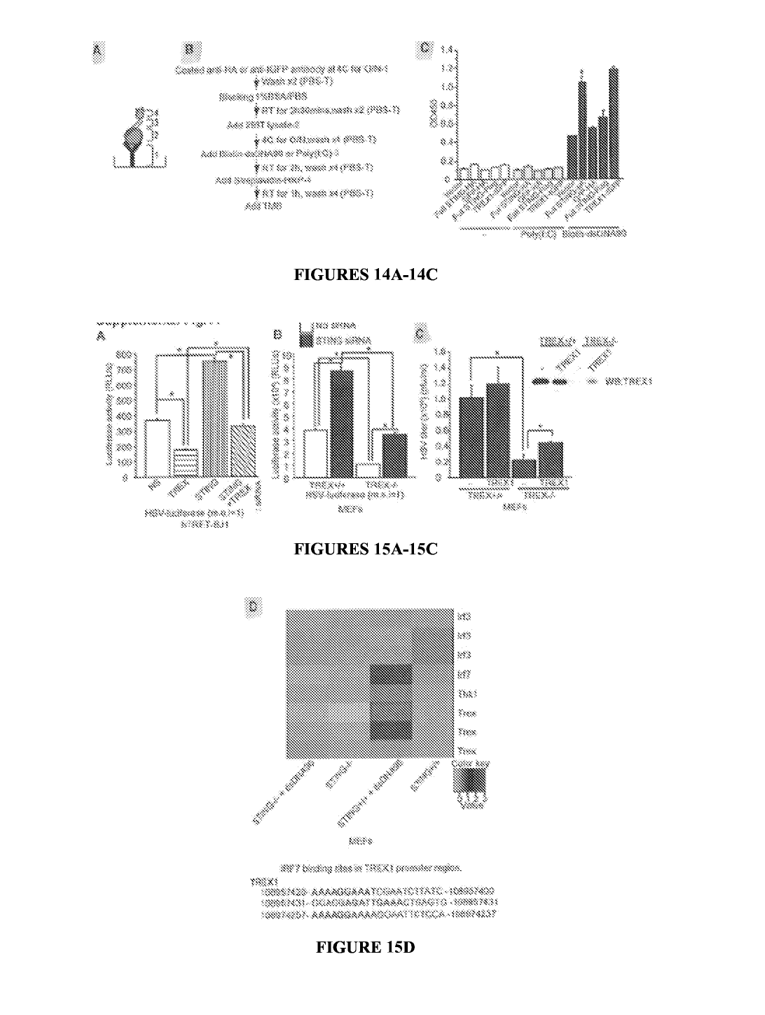

To further evaluate the possibility that STING itself could associate with DNA species, 293T cells were transfected with STING and after cell lysis observed that the C-terminal region of STING (aa 181-349) could be precipitated using biotin-labeled dsDNA90 (FIG. 2A). The N-terminal region of STING (aa 1-195) and three similarly HA-tagged controls (GFP, NFAR1 and IPS1) did not associate with dsDNA90. The DNA binding exonuclease TREX1 served as a positive control. A further series of extensive studies indicated that amino acid region 242-341 of STING was likely responsible for binding dsDNA since STING variants lacking this region failed to associate with nucleic acid (FIGS. 2B-D). In vitro expressed STING also bound to dsDNA under high salt and high detergent conditions (except for those variants similarly lacking region 242-341) (FIGS. 11A-F). Further evidence that STING could complex with dsDNA, likely as a dimer, was achieved by transfecting biotin-labeled dsDNA90 into hTERT's and treating such cells with a reversible cross-linking reagent (DSS) or UV light. In both treatment cases STING was observed to retain its association with DNA after cell lysis (FIG. 2E and FIGS. 12A-G). RNAi knockdown of STING in hTERT cells eliminated the observed binding and STING-DNA complexes were also only observed in wild type MEFs (^{+/+}) but not MEFs lacking STING (^{-/-}) (FIGS. 12A-G). It was similarly confirmed that HSV1, cytomegalovirus (CMV) as well as adenovirus (ADV) related dsDNA Competition experiments suggested that STING also could bind to ssDNA (ssDNA90) as well as dsDNA, but not dsRNA (FIG. 2F). This was confirmed by expressing STING in vitro and observing association with ssDNA90 (FIG. 2G). All STING variants analyzed lacked the ability to activate the type IFN promoter in 293T cells (FIG. 2H). dsDNA was also transfected into hTERT or MEFs cells and treated with formaldehyde to cross-link cellular proteins to the nucleic acid. Subsequent CHIP analysis following STING pull down, further confirmed that transfected DNA can directly associate with STING as determined using dsDNA90 specific primers (FIGS. 2I and 2J). It was observed that STING could bind to biotin-labeled DNA in ELISA assays (FIGS. 14A-C). The data indicated that ssDNA and dsDNA-mediated innate signaling events were dependent on STING and evidence that STING itself was able to complex to these nucleic acid structures to help trigger these events.

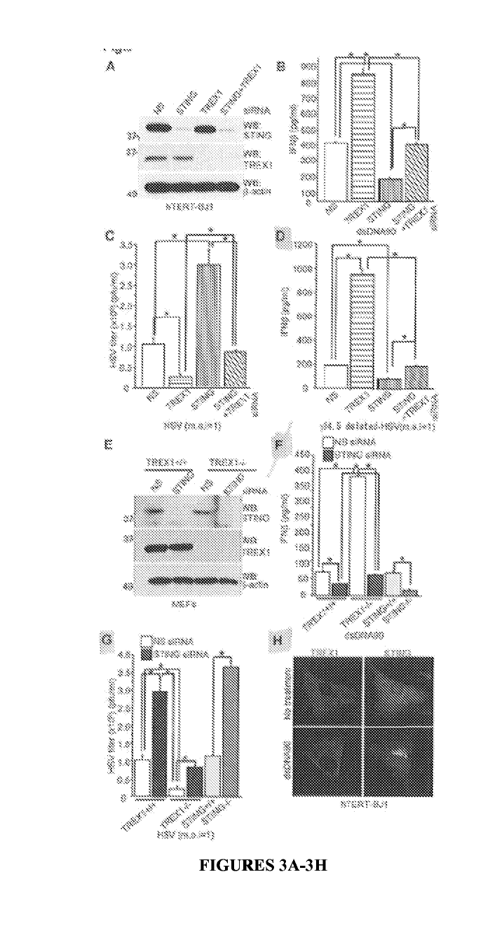

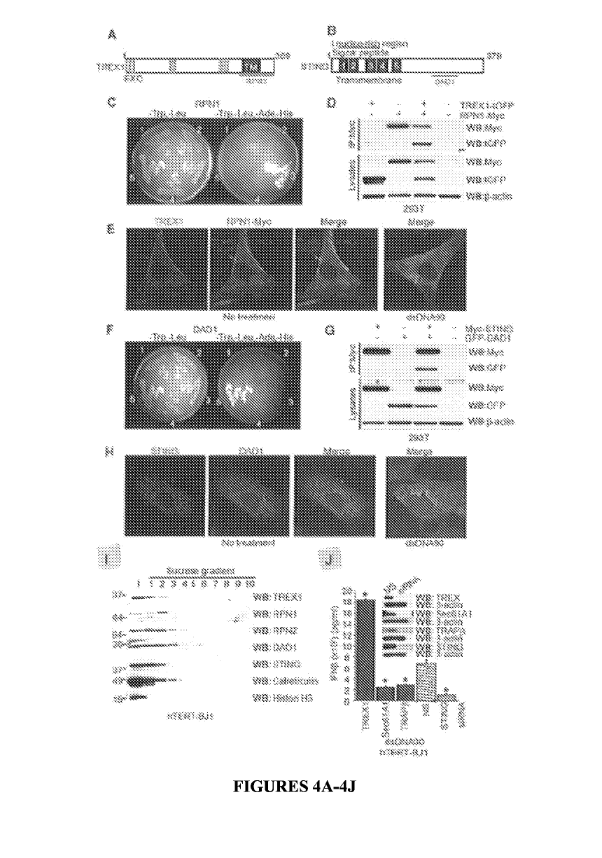

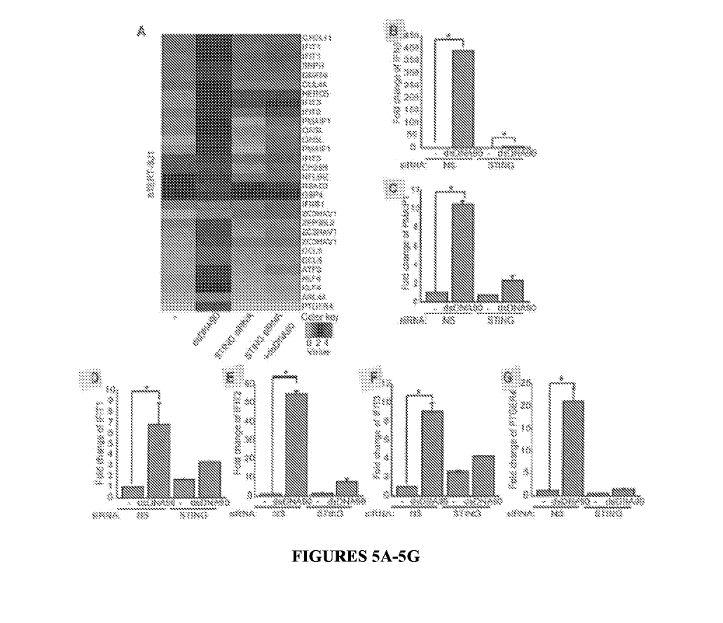

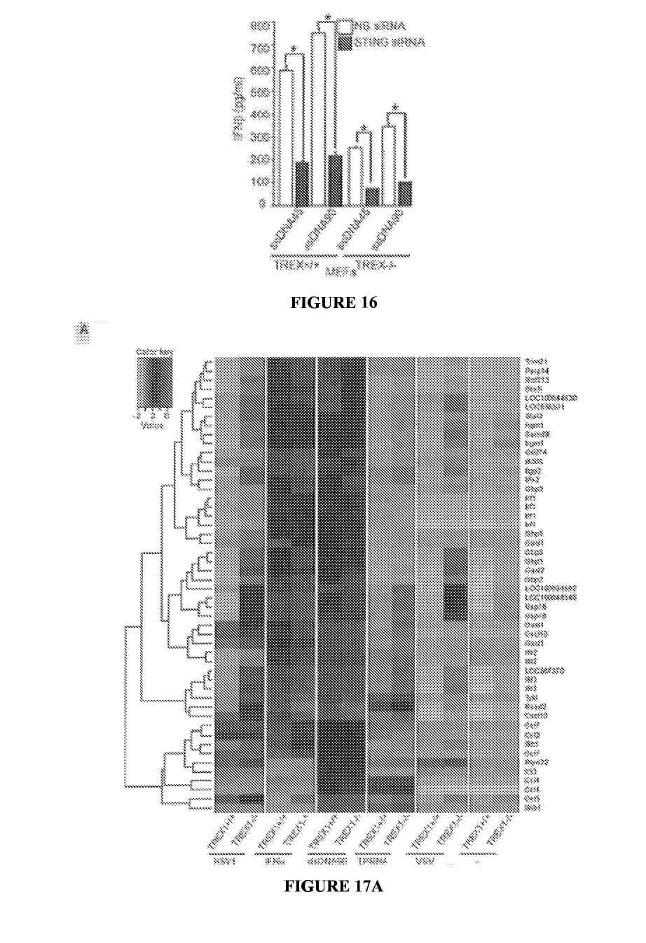

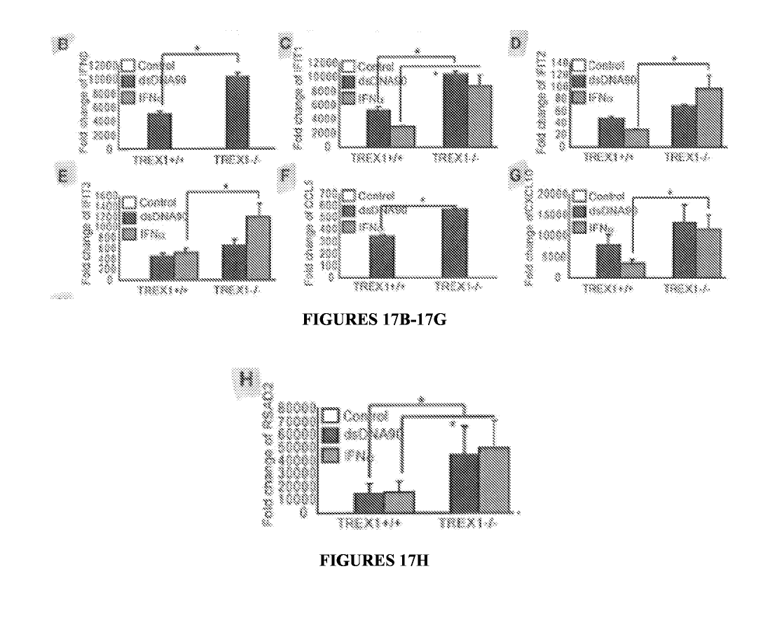

TREX1, a 3′->5′ DNA exonuclease is also an ER associated molecule, and important for degrading checkpoint activated ssDNA species that could otherwise activate the immune system. RNAi used to silence TREX1 in hTERT cells significantly increased STING-dependent, production of type I IFN by dsDNA90 (FIGS. 3A and 3B). Concomitantly, the replication of the dsDNA virus HSV1 was greatly reduced in hTERT cells lacking TREX1, likely due to the elevated production of type I IFN and antiviral IFN stimulated genes (ISG's) (FIGS. 3C and D). Luciferase expression from a recombinant HSV-expressing the luciferase gene was also significantly lower in hTERT infected cells treated with RNAi to silence TREX1 (FIGS. 15A-D). These observations were extended by using TREX1 deficient MEFs, which similarly indicated that cytoplasmic dsDNA-dependent gene induction was greatly elevated in the absence of TREX1 and that HSV1 replication was significantly reduced (FIGS. 3E-G). To determine if STING was responsible for the elevated production of type I IFN observed in the absence of TREX1, STING was silenced in TREX1 lacking hTERTs or TREX1^{-/-} MEFs and treated these cells with cytoplasmic dsDNA or HSV1. The results indicated greatly reduced type I IFN production in TREX1 deficient cells (both hTERTs and MEFs) lacking STING indicating that the elevated levels of type I IFN observed in the absence of TREX1 are STING-dependent (FIGS. 3A-F). Similarly, it was noted that RNAi knockdown of STING in TREX^{-/-} MEFs also eliminated ssDNA90-mediated type I IFN production and innate gene stimulation (FIG. 16). Confocal analysis confirmed that TREX1 and STING colocalized in the ER (FIG. 3H). However, cytoplasmic dsDNA did not potently induce the trafficking of TREX1 from the ER to perinuclear regions similar to STING (FIG. 3H). Thus, it was not observed that STING and TREX1 interacted robustly, as determined by coimmunpreciptation analysis. Neither was a dramatic difference noticed in the expression of STING-dependent genes in TREX1+/+ or ^{-/-} MEFs under non-stimulated conditions (FIGS. 17A-H). However, TREX1 is a dsDNA-induced gene, which was confirmed and can be upregulated in a STING-dependent manner (FIGS. 17A-H). Thus, it is plausible that dsDNA species complex with STING and accessory molecules to mediate trafficking and downstream signaling events that activate the transcription factors IRF3/7 and NF-κB, responsible for the induction of primary innate immune genes including TREX1. The evidence indicates that STING-activated TREX1 resides in the ER region to degrade activator dsDNA and repress cytoplasmic dsDNA signaling in a negative-feedback manner. Thus, TREX1 is a negative regulator of STING.

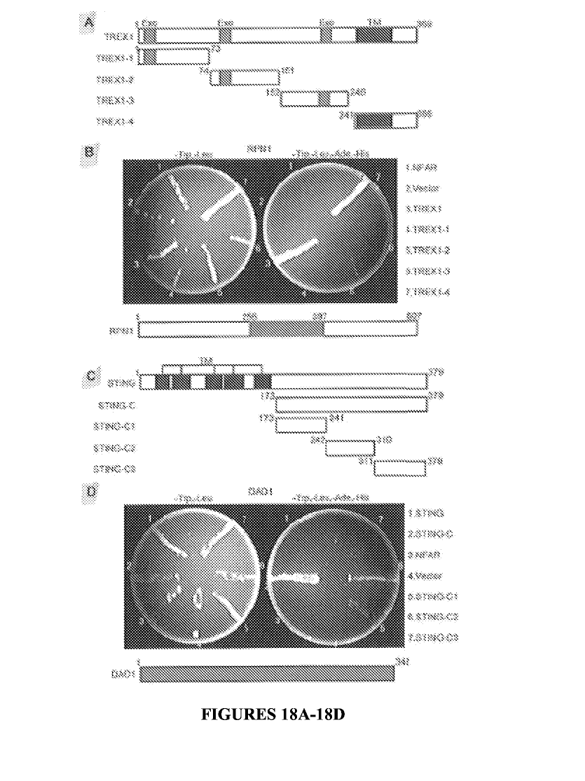

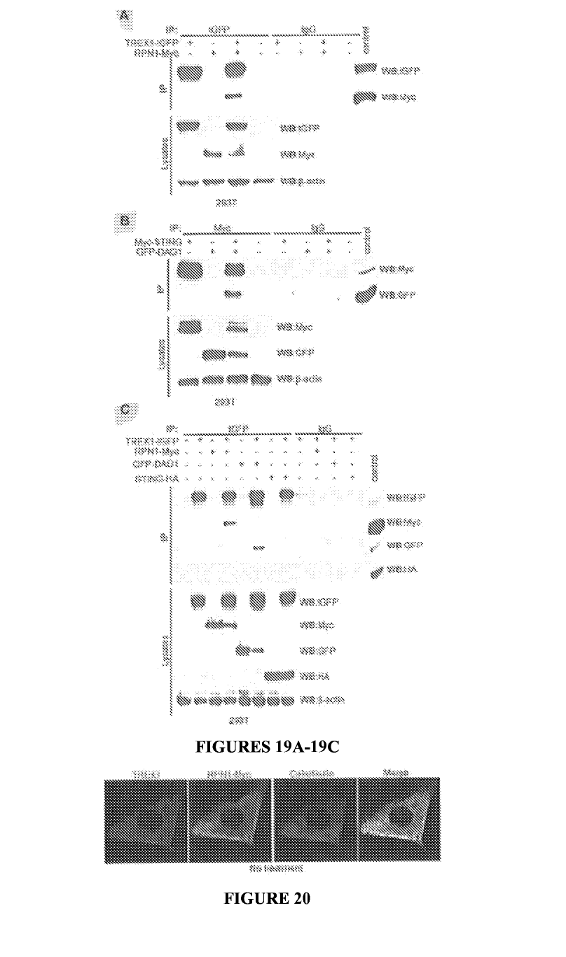

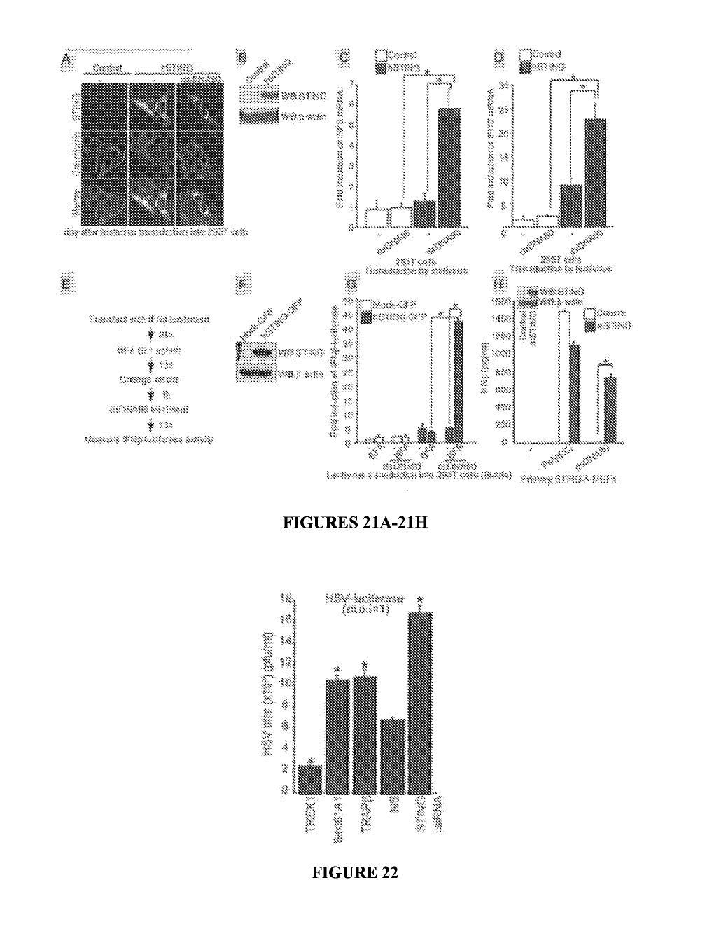

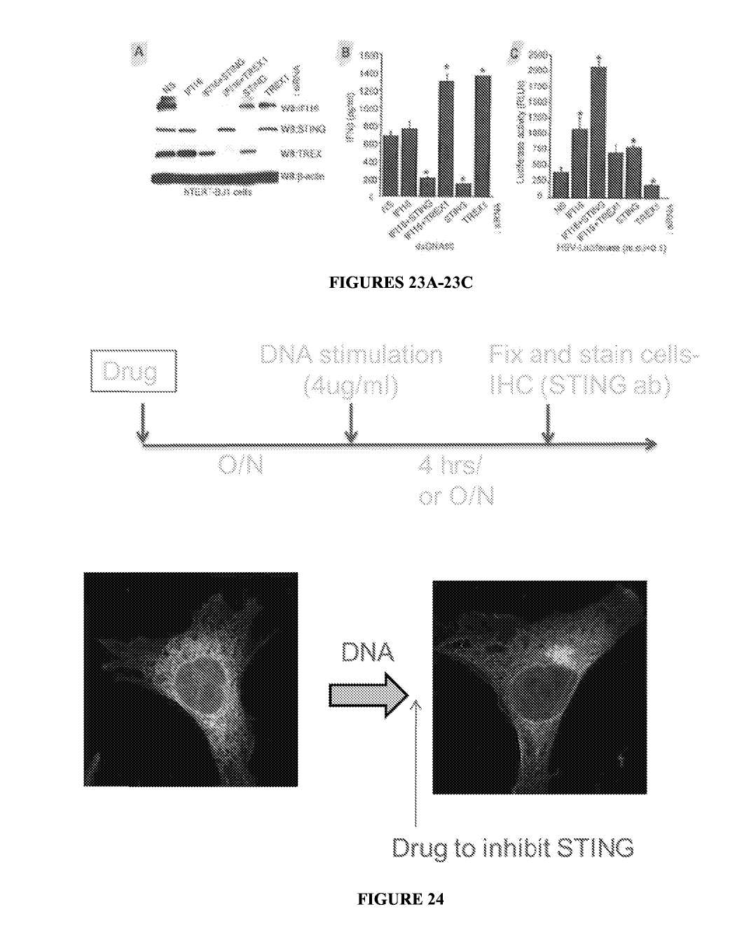

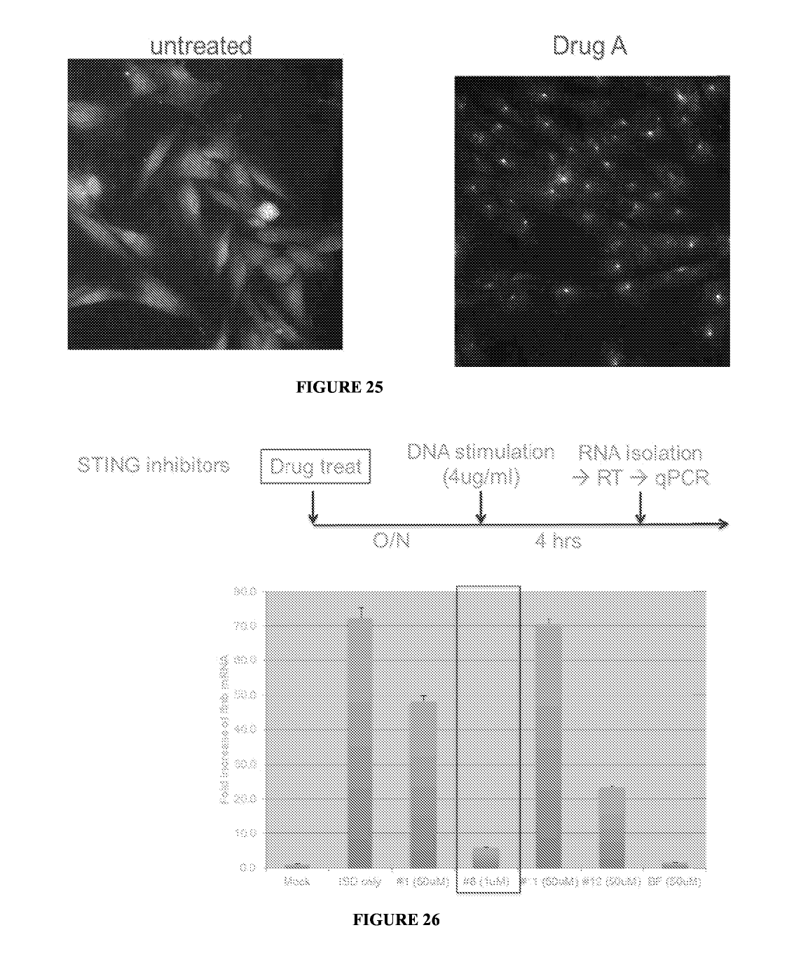

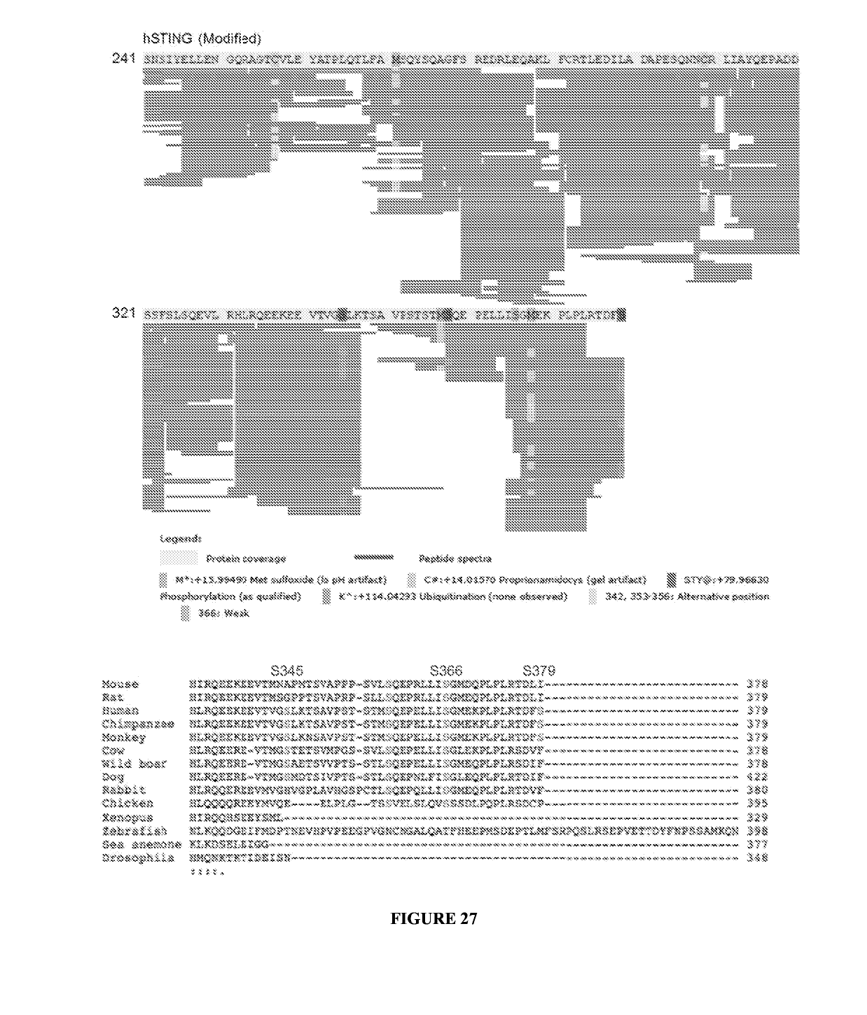

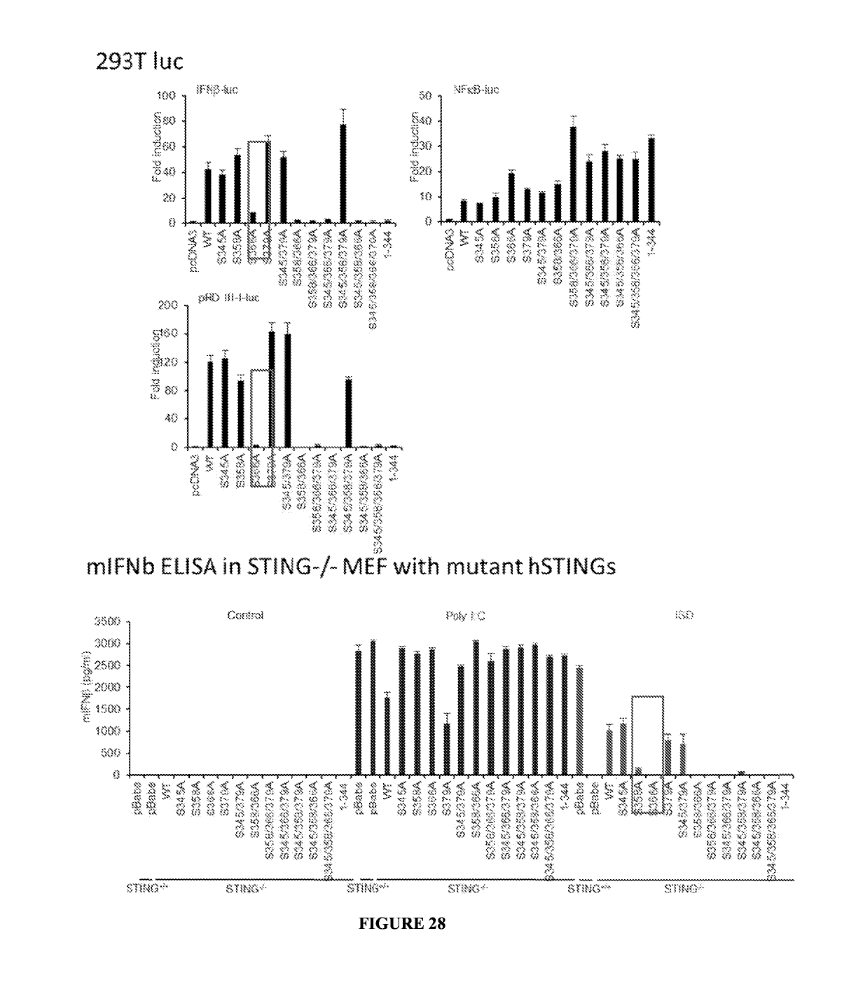

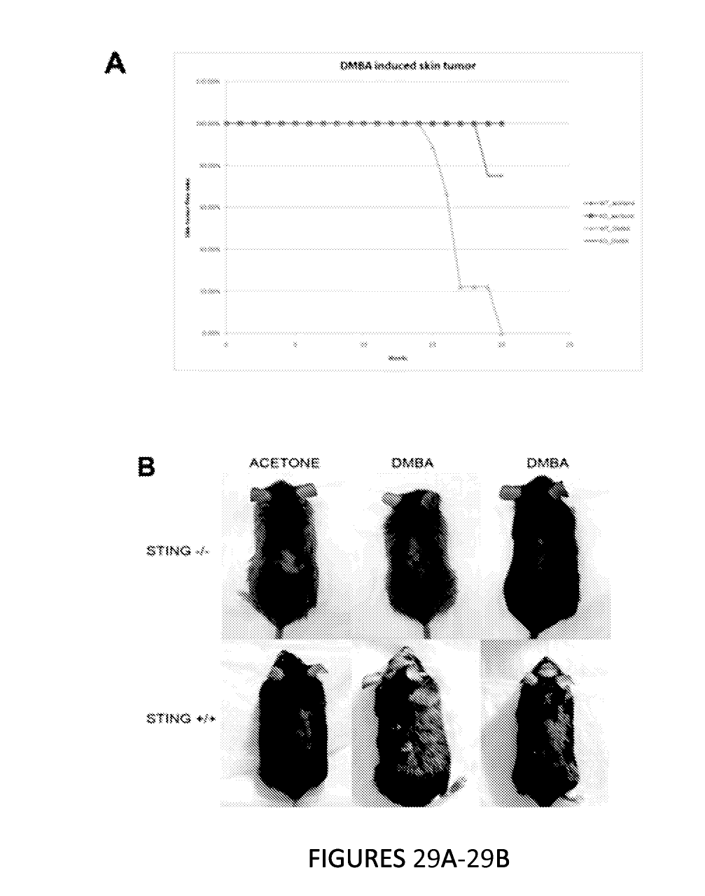

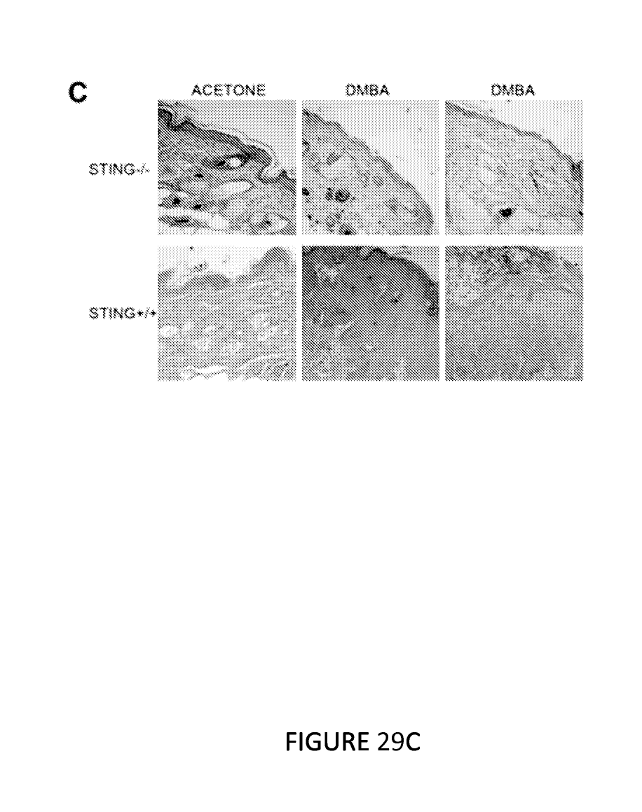

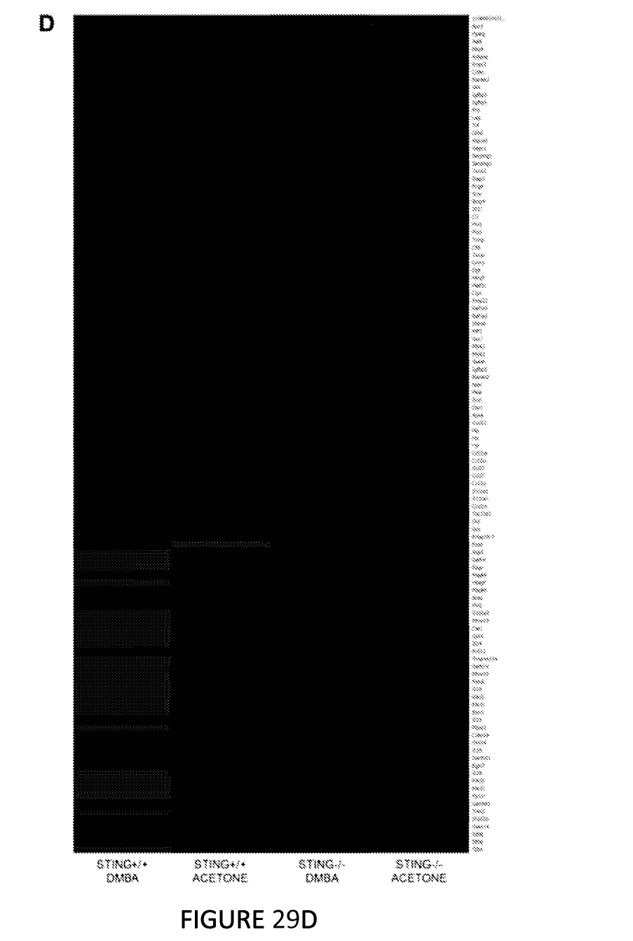

The data herein demonstrated that STING resides in the ER as part of the translocon complex, associating with translocon associated protein β (TRAPβ). The translocon complex includes Sec61 α ρ and γ coupled with TRAP α ρ, γ and δ, which can attach to ribosomes. Secretory and membrane proteins are translocated into the ER for proper folding and glycosylation prior to being exported. To identify TREX1 binding partners full length TREX1 was used as bait in a two hybrid yeast screen. The results indicated that TREX1 recurrently interacted with a protein referred to as Ribophorin I (RPN1), a 68 kDa type I transmembrane protein and member of the oligosaccharyltransferase (OST) complex (FIGS. 4A-E; FIGS. 18A-D). The OST complex catalyses the transfer of mannose oligosaccharides onto asparagines residues of nascent polypeptides as they enter the ER through the translocon. At least seven proteins include the OST complex including RPN1, RPN2, OST48, OST4, STT3A/B, TUSC3 and DAD1. Significantly, a similar screen using STING as bait determined that STING could associate also with DAD1 (defender against apoptotic cell death), a 16 kDa transmembrane protein (FIGS. 14F-H). Further analysis of these associations using yeast-two hybrid approaches indicated that the C-terminal region of TREX1 comprising its transmembrane region (amino acids 241-369) was responsible for binding to amino acids 258-397 of RPN1 (FIGS. 18A-D). Further, amino acids 242-310 of STING were accountable for association with full length DAD1 (FIGS. 18A-D). Coimmunoprecipitation studies confirmed the interaction of these molecules (FIGS. 4D and 4G and FIGS. 29A-C). Further co-immunoprecipitation experiments indicated the association of TREX1 with DAD1 (FIGS. 19A-C). Confocal analysis confirmed that TREX1 and RPN1 co-localized in the cell though did not traffic in response to cytoplasmic dsDNA (FIG. 4E and FIG. 20). Similarly, STING and DAD1 colocalized in the ER of the cell although the latter molecule did not accompany STING to endosomal compartments in the presence of cytoplasmic dsDNA (FIG. 4H). Cellular microsome compartments comprising the ER were isolated by fractionation and examined by sucrose gradient analysis. This study indicated that TREX1 and STING cofractionated with the ER markers RPN1 and RPN2, DAD1 and calreticulin, but not nuclear histone H3, confirming that their subcellular localization is indistinguishable from components of the translocon/OST complex (FIG. 4I). Thus, TREX1 is targeted to the OST/translocon complex of the ER, that includes STING, and this association occurs through association with RPN1, although the TM region of TREX1 was found to be involved in TREX1's localization to the ER. To identify whether members of the OST, TRAP or SRP (signal recognition peptide) complex influenced dsDNA-dependent signaling, an RNAi screen was carried out to silence the expression of these components. However, aside from repressing STING (essential for DNA-mediated type I IFN production; FIGS. 21A-H) and TREX1, which greatly elevated type I IFN production, only Sec61 α and TRAPβ silencing significantly affected signaling and HSV1 replication, evidencing a role for these translocon members in controlling this pathway (FIG. 4J and FIG. 22). Silencing of IFI16, also implicated in cytoplasmic DNA sensing, was observed to not robustly suppress dsDNA-dependent signaling, at least in hTERT cells (FIGS. 23A-C). Neither did loss of IFI16 rescue augmented IFN-production by dsDNA in the absence of TREX1, similar to loss of STING (FIGS. 23A-C). However, reduced IFI16 enabled more proficient HSV1 gene expression confirming an important role for this molecule in influencing viral replication. Silencing of RPN1 or 2 also lead to an increase in HSV1 gene expression but did not significantly affect type I IFN production either, evidencing that these components of the OST may be predominantly involved in N-glycosylation.

The data evidences that STING can complex with cytoplasmic intracellular ssDNA and dsDNA, which can include plasmid-based DNA and gene therapy vectors, can regulate the induction of a wide array of innate immune genes such as type I IFN, the IFIT family, and a variety of chemokines important for antiviral activity and for initiating adaptive immune responses. STING activation facilitates the escort of TBK1 to clathrin covered endosomal compartments plausibly to activate IRF3/7 by mechanisms that remain to be fully clarified. TREX1 appears present in low levels in the cell and is itself inducible by STING. After translation, TREX1 localizes to the OST complex in close proximity to unactivated STING (which also resides in the OST/translocon complex) where presumably it degrades DNA species that can otherwise provoke STING action. Components of the translocon/OST complex, which now involve STING and TREX1, regulate cytoplasmic ssDNA and dsDNA-mediated innate immune signaling. Since loss of TREX1 manifests autoimmune disorders through elevated type I IFN production, it is possible that these diseases are induced through STING activity.

### Example 2

### STING Modulators

Drug libraries were screened to identify agents that modulate STING expression, function, activity, etc. FIG. 24 shows the steps of a STING cell based assay.

The libraries included, BioMol ICCB known Bioactives Library, 500 targets; LOPAC1280™ Library of Pharmacologically-Active Compounds; Enzo Life Sciences, Screen-Well™ Phosphatase Inhibitor Library, 33 known phosphatase inhibitors;

MicroSource Spectrum Collection 2000 components, 50% drug components, 30% natural products, 20% other bioactive components; EMD: InhibitorSelect™ 96-Well Protein Kinase Inhibitor Library I, InhibitorSelect™ 96-Well Protein Kinase Inhibitor Library II, InhibitorSelect™ 96-Well Protein Kinase Inhibitor Library IIIa; Kinase Library B Kinase TrueClone collection; Kinase Deficient TrueClone collection.

The results showed that one drug (termed ‘Drug A”) induced STING trafficking (FIG. 60). Another drug (termed “drug X”) inhibited IFNβ mRNA production (FIG. 61).

Table 2: The following were identified as STING inhibitors:

| Name | Chemical Name | Description |
|---|---|---|
| Diclofenac sodium | 2-[(2,6- | Cyclooxygenase inhibitor; |
| | Dichlorophenyl)amino]benzeneacetic | anti-inflammatory |
| | acid sodium | |
| R(−)-2,10,11- | R(−)-2-Hydroxyapomorphine | D2 dopamine receptor |
| Trihydroxyaporphine | hydrobromide | agonist |
| hybrobromide | | |
| Dipropyldopamine | | Dopamine receptor agonist |
| hydrobromide | | |
| 2,2′-Bipyridyl | alpha,alpha′-Bipyridyl | Metalloprotease inhibitor |
| (±) trans-U-50488 | trans-(±)-3,4-Dichloro-N-methyl-N-[2- | Selective kappa opioid |
| methanesulfonate | (1-pyrrolidinyl)-cyclohexyl]- | receptor agonist. |
| | benzeneacetamide | |
| | methanesulfonate | |
| SP600125 | Anthrapyrazolone; 1,9- | Selective c-Jun N-terminal |
| | Pyrazoloanthrone | kinase (c-JNK) inhibitor. |
| Doxazosin mesylate | 1-(4-amino-6,7-dimethoxy-2- | alpha1 adrenoceptor blocker |
| | quinazolinyl)-4-[4-(1,4-benzodioxan- | |
| | 2-yl)carpiperazin-1-yl)]-6,7- | |
| | dimethoxyquinazoline mesylate | |
| Mitoxantrone | 1,4-Dihydroxy-5,8-bis([2-([2- | DMA synthesis inhibitor |
| | hydroxyethyl]amino)ethyl]amino)- | |
| | 9,10-anthracenedione | |
| MRS 2159 | | P2X1 receptor antagonist |
| Nemadipine-A | 1,4-Dihydro-2,6-dimethyl-4- | An L-type calcium channel |
| | (pentafluorophenyl)-3,5- | alpha1-subunit antagonist. |
| | pyridinedicarboxylic acid diethyl | |
| | ester | |
| (±)-PPHT hydrochloride | (±)-2-(N-Phenylethyl)-N- | Potent D2 dopamine |
| | propyl)amino-5-hydroxytetralin | receptor agonist |
| | hydrochloride | |
| SMER28 | 6-Bromo-N-2-propenyl-4- | Small molecule modulator of |
| | quinazolinamine | mammalian autophagy. |
| Quinine sulfate | | K+ channel blocker; |
| | | antimalarial, anticholinergic, |
| | | antihypertensive and |
| | | hypoglycemic agent; alkaloid |
| | | isolated from the bark of the |
| | | Cinchona family of South |
| | | American trees |
| (+)-Quisqualic acid | L(+)-alpha-Amino-3,5-dioxo-1,2,4- | Active enantiomer of |
| | oxadiazolidine-2-propanoic acid | quisqualic acid; excitatory |
| | | amino acid at glutamate |
| | | receptors; anthelmentic |
| | | agent |

Activators of STING included dihydroouabain and BNTX maleate salt hydrate.

### Example 3

### STING Manifests Self DNA-Dependent Inflammatory Disease

Bone marrow derived macrophages (BMDM) were obtained from Sting_{+/+} and Sting^{-/-} mice and transfected them with 90 base pair dsDNA (dsDNA90) to activate the STING pathway, or with apoptotic DNA (aDNA) derived from dexamethasone (Dex)-treated thymocytes. It was observed that both types of DNA potently induced the production of IFNβ in BMDM and conventional dendritic cells (BMDC's) in a STING dependent manner. DNA microarray experiments confirmed that aDNA triggered STING-dependent production of a wide array of innate immune and inflammatory related cytokines in BMDM such as IFNβ as well as TNFα (Table 3). These data were confirmed by measuring cytokine production in Sting_{+/+} or Sting^{-/-} BMDM treated with aDNA. Thus, STING can facilitate apoptotic DNA-mediated pro-inflammatory gene production in BMDM's as well as BMDC's.

Table 3 shows the gene expression of higher expressed genes in BMDM treated with apoptic DNA (aDNA).

| | BMDM (STING +/+) | | | BMDM (STING −/−) | | |
|---|---|---|---|---|---|---|
| | Signal | | Fold | Signal | | Fold |
| Symbol | Mock | aDNA | Increase | Mock | aDNA | Increase |
| Cxcl9 | 6.338 | 1.281 | 33.303 | −1.102 | −1.524 | 1.339 |
| Ifnb1 | 5.594 | 0.728 | 29.162 | −0.846 | −0.916 | 1.049 |
| Ifna11 | 4.548 | 0.085 | 22.051 | −0.032 | −0.064 | 1.022 |
| Ifn [symbol] | 4.403 | −0.038 | 21.718 | −0.083 | −0.027 | −1.029 |
| Ccl5 | 4.651 | 0.403 | 20.947 | −1.654 | −3.732 | 2.481 |
| Fam26f | 4.493 | 0.597 | 14.888 | −2.052 | −4.000 | −1.008 |
| Ifna6 | 3.831 | 0.020 | 14.044 | −1.819 | −3.705 | −1.019 |
| AA467197 | 2.486 | −1.303 | 13.822 | 0.000 | −0.342 | 1.268 |
| Ifna2 | 3.665 | −0.055 | 13.179 | 0.000 | −0.129 | 1.094 |
| Ifna5 | 3.609 | −0.051 | 12.637 | −0.068 | −0.027 | −1.028 |
| [symbol] | 2.242 | −1.183 | 10.723 | −1.409 | −2.737 | 2.116 |
| H [symbol] | 3.416 | 0.037 | 10.400 | −0.820 | −2.230 | 1.016 |
| Hdc | 3.181 | 0.120 | 8.346 | −0.094 | −0.214 | 1.086 |
| Oasl1 | 3.747 | 0.688 | 8.330 | −0.715 | −0.766 | 1.036 |
| Gm14446 | 4.130 | 1.078 | 8.294 | −1.742 | −3.029 | 1.109 |
| Ifi205 | 3.805 | 0.788 | 8.093 | −1.931 | −3.199 | 1.051 |
| Oasl1 | 4.202 | 1.108 | 8.021 | −0.565 | −1.865 | 1.036 |
| [symbol] | 4.597 | 1.505 | 8.011 | −1.931 | −3.199 | 1.013 |
| LOC109844782 | 2.807 | 0.041 | 7.281 | 0.470 | −1.334 | 3.511 |
| Gbp5 | 3.852 | 1.063 | 6.913 | −2.076 | −3.180 | 1.125 |
| Gja4 | 2.989 | 0.203 | 6.895 | 0.241 | −0.840 | −1.000 |
| Oasl1 | 4.312 | 1.547 | 6.795 | −0.565 | −1.865 | 1.602 |
| Il33 | 2.754 | −0.010 | 6.792 | −2.038 | −3.039 | 1.001 |
| Plekha4 | 3.399 | 0.672 | 6.623 | 0.750 | −0.248 | −1.011 |
| C [symbol] | 3.454 | 0.753 | 6.501 | −1.614 | −2.804 | 1.032 |
| Hap1 | 2.844 | 0.161 | 6.530 | 0.624 | −0.302 | 1.045 |
| Gm12597 | 2.570 | −0.072 | 6.243 | −0.659 | −1.629 | 1.013 |
| Serpina3f | 2.607 | 0.022 | 6.000 | 0.644 | −0.261 | 1.203 |
| Cxcl1 | 2.210 | −0.373 | 5.884 | −0.783 | −1.681 | 4.791 |
| LOC1 [symbol] 44573 | 2.532 | 0.020 | 5.703 | −0.485 | −1.372 | −1.084 |
| Ifna4 | 2.468 | −0.032 | 5.658 | 0.541 | −0.334 | −1.014 |
| Upp1 | 2.188 | −0.275 | 5.515 | 0.460 | −0.414 | 1.395 |
| Ccl8 | 2.579 | 0.122 | 5.490 | −0.141 | −0.234 | 1.066 |
| Atm | 2.549 | 0.101 | 5.455 | −0.136 | −0.127 | −1.006 |
| Phf11 | 3.545 | 1.113 | 5.397 | 0.606 | −0.244 | 1.303 |
| G [symbol] 7 | 1.364 | −1.043 | 5.305 | 1.452 | 0.627 | −1.083 |
| [symbol] 2 | 2.282 | −0.099 | 5.203 | 0.081 | −0.718 | 1.083 |
| Phfl1 | 3.572 | 1.233 | 5.080 | 1.484 | 0.708 | 1.008 |
| Lif | 2.209 | −0.127 | 5.049 | 0.292 | −0.485 | −1.033 |
| Lcn2 | 2.408 | 0.080 | 5.020 | 0.440 | −0.322 | 1.287 |

[symbol]

indicates data missing or illegible when filed

To determine if STING played a role in DNase II related inflammatory disease, STING and/or DNase II was knocked down in THP1 cells or BMDM using RNAi and it was noticed that loss of DNase II facilitated the upregulation of cytokines, including type I IFN, in response to aDNA in a STING-dependent manner. Since DNase II^{-/-} mice usually die before birth, DNase II^{-/-}, Sting^{-/-}, or Sting^{-/-} DNase II^{-/-} DKO 17 day embryos (E17 days) were analyzed. Genotyping analysis, including RT-PCR and immunoblot confirmed that the embryos lacked Sting, DNase II or both functional genes. It was observed that DNase II^{-/-} embryos exhibited anemia, as described above, which was in significant contrast to Sting^{-/-} DNase II^{-/-} DKO embryos or controls which noticeably lacked this phenotype. Lethal anemia has been reported to be due to type I IFN inhibition of erythropoiesis during development. It was subsequently observed by hematoxylin and eosin staining that the livers of DNase II^{-/-} embryos contained numerous infiltrating macrophages full of engulfed apoptotic cells responsible for producing high levels of cytokines. In contrast to control mice, the livers of Sting^{-/-} DNase II^{-/-} embryos exhibited a similar phenotype. Analysis of fetal livers by TUNEL (terminal deoxynucleotidyl transferase-mediated dUTP biotin nick end-labeling) confirmed that the Sting^{-/-} DNase II^{-/-} embryos and DNase II-deficient but not wild-type fetal livers contained numerous large inappropriately digested dying cells. In vitro analysis has indicated that macrophages from the embryos of of wild-type or DNase II^{-/-} mice engulf apoptotic cells adequately. However, while the DNA of the engulfed apoptotic cells is efficiently degraded in the lysosomes of wild-type macrophages, DNase II^{-/-} macrophages accumulate engulfed nuclei and cannot digest DNA. This event leads to the stimulation of innate immune signaling pathways and production of autoimmune related cytokines. Given this, the ability of embryonic liver derived macrophages that lacked both DNase II and STING were evaluated as to whether they to engulf apoptotic cells and digest DNA. It was noted that Sting^{-/-} DNase II^{-/-} macrophages, similar to DNase II^{-/-} macrophages, were not able to digest the engulfed nuclei from dexamethasone treated apoptotic thymocytes compared to control macrophages taken from wild type or Sting^{-/-} mice. Thus, macrophages harvested from the livers of Sting^{-/-} DNase II^{-/-} embryonic mice similarly exhibit an inability to digest engulfed apoptotic cells, analogous to DNase II^{-/-} macrophages.

The above analysis was complemented with analyzing mRNA expression levels in the livers of the embryonic mice. This study indicated very little inflammatory gene production in the livers of wild type or Sting^{-/-} embryos. However, it was observed that the livers of DNase II^{-/-} embryos contained abnormally high levels of cytokine related mRNA. Significantly, the livers of Sting^{-/-} DNase II^{-/-} mice had dramatically reduced levels of innate immune gene expression activity compared to DNase II^{-/-} mice. These results were confirmed by analyzing the mRNA expression levels of select innate immune genes in embryonic livers by RT-PCR. For example, the production of IFNβ was reduced several fold in Sting^{-/-} DNase II^{-/-} mice compared to DNase II^{-/-} mice. The production of key interferon-stimulated genes (ISG's) such as the 2′-5′ oligoadenylate synthetases (OAS), interferon-induced proteins with tetratricopeptide repeats (IFITs) interferon-inducible protein 27 (IFI27) and ubiquitin-like modifier (ISG15) were also dramatically reduced. Pro-inflammatory cytokines such as TNFα and IL1β were also decreased in the embryonic livers of Sting^{-/-} and Sting^{-/-} DNase II^{-/-} compared to DNase II^{-/-} mice. While the production of innate immune genes was dramatically suppressed in the absence of STING, the presence of some genes remained slightly elevated in Sting^{-/-} DNase II^{-/-} mice, albeit in low levels as determined by anay analysis, which may be due to variation in mRNA expression between the animals analyzed, or perhaps due to the stimulation of other pathways. Many of these genes are regulated by NF-κB and interferon regulatory factor (IRF) pathways. The function of these transcription factors was thus evaluated in Sting^{-/-} DNase II^{-/-} or control murine embryonic fibroblasts (MEFs), developed from 14 day embryos (E14 days). Principally, a defect was observed in NF-κB activity (p65 phosphorylation) in Sting^{-/-} DNase II^{-/-} MEFs when exposed to cytoplasmic DNA. The same defect was obtained in Sting^{-/-} DNase II^{-/-} BMDM when exposed to apoptotic DNA as well as cytoplasmic DNA. This was confirmed by noting that NF-κB as well as IRF3 also failed to translocate in Sting^{-/-} DNase II^{-/-} MEF's but not in control MEFs following exposure to dsDNA. Thus, STING is likely responsible for controlling self DNA-induced inflammatory cytokine production that is responsible for causing lethal embryonic erythropoiesis.

To extend of the importance of STING in mediating self-DNA-facilitated lethal erythropoiesis, it was evaluated whether DNase II^{-/-} mice could be born in the absence of STING. Significantly, it was observed that DNase II^{-/-} mice were born, with apparent Mendelian frequency, when crossed onto a Sting^{-/-} background. PCR genotyping, Northern blot, RT-PCR and immunoblot analysis confirmed DNase II and STING deficiency in the progeny mice. Sting^{-/-} DNase II^{-/-} double knockout mice (DKO) appeared to grow normally and exhibited similar size and weight compared to control mice although it was noted that Sting^{-/-} mice were somewhat larger for reasons that remain unclear. Preliminary immunological evaluation also indicated that the Sting^{-/-} DNase II^{-/-} DKO animals shared a similar CD4⁺/CD8⁺ profile similar to Sting^{-/-} and and wild type mice, although the DKO's were noted to develop splenomegaly as they aged. Splenomegaly was also noted in surviving DNase II deficient mice that lacked type I IFN signaling (DNase II^{-/-} Ifnar1^{-/-} mice) and has been reported to be due to enlargement of the red pulp. However, analysis of serum from Sting^{-/-} DNase II^{-/-} mice indicated no detectable abnormal cytokine production compared to control mice at 8 weeks of age, due to the general low immeasurable levels of cytokines produced. Through these studies it was noted in vitro that Sting^{-/-} DNase II^{-/-} macrophages, similar to DNase II^{-/-} macrophages, were not able to digest the engulfed nuclei from apoptotic thymocytes (Dex⁺) compared to control macrophages taken from wild type or Sting^{-/-} mice. The accumulation of undigested DNA in DNase II^{-/-} Sting^{-/-} macrophages was less pronounced when WT thymocytes were used as targets (Dex⁻). Thus, BMDM derived from Sting^{-/-} DNase II^{-/-} mice are also incapable of digesting DNA from apoptotic cells, although in contrast to DNase II^{-/-} BMDM do not produce inflammatory cytokine responses.

While DNase II mediated embryonic lethality can be avoided by crossing DNase II^{+/-} mice with type I IFN defective Ifnar1^{-/-} mice, the resultant progeny suffer from severe polyarthritis approximately 8 weeks after birth (arthritis score of 2) since undigested DNA activates innate immune signaling pathways and triggers the production of inflammatory cytokines such as TNFα. Significantly, it was noted that Sting^{-/-} DNase II^{-/-} mice did not manifest any signs of polyarthritis following birth. Arthritis scores remained at approximately zero (no score) in the Sting^{-/-} DNase II^{-/-}, up to 12 months of age in contrast to reported DNase II^{-/-} Ifnar1^{-/-} mice which exhibited an arthritis score of up to 7 after a similar period. While H&E and TUNEL staining of spleen and thymus tissues of DNase II^{-/-} Sting^{-/-} mice illustrated the presence of infiltrating macrophages that also contained apoptotic DNA, histology of joints from 6 month old Sting^{-/-} DNase II^{-/-} mice exhibited normal bone (B) synovial joint (S) and cartilage (C) structure with no evidence of pannus infiltration in the joint structure. Levels of TNFα, IL1β and IL6 from sera of Sting^{-/-} DNase II^{-/-} mice remained at low levels as predicted from our array analysis of BMDM that lacked STING (Table 3). Neither was there evidence of CD4, CD68 or TRAP positive cells infiltration within the joints of Sting^{-/-} DNase II^{-/-} mice. Analysis of the serum of Sting^{-/-} DNase II^{-/-} mice also indicated no elevated levels of Rheumatoid Factor (RF), anti-dsDNA antibody or MMP3. Thus, loss of STING eliminates pro-inflammatory cytokine production responsible for self DNA-mediated polyarthritis.

STING is responsible for inflammatory disease such as for example, Aicardi-Goutieres syndrome (AGS). AGS is genetically determined encephalopathy and is characterized by calcification of basal ganglia and white matter, demyelination. High levels of lymphocytes and type I IFN in cerebrospinal fluid. The features mimic chronic infection. Serum levels of type I IFN are also raised in autoimmune syndrome systemic lupus erythromatosis (SLE). AGS is caused by mutations in 3′-5′ DNA exonuclease TREX1. Loss of TREX1 function-DNA species accumulates in the ER of cells and activates cytoplasmic DNA sensors (STING). TREX1 digests this DNA source (housekeeping function) to prevent innate immune gene activation.

Given that STING seems responsible for inflammatory disease in mice defective in apoptosis, it was next evaluated whether other types of self-DNA triggered disease occurred through activation of the STING pathway. For example, patients defective in the 3′ repair exonuclease 1 (Trex1) suffer from Aicardi-Goutieres Syndrome (AGS) which instigates lethal encephalitis characterized by high levels of type I IFN production being present in the cerebrospinal fluid. Trex1-deficient mice exhibit a median life span of approximately 10 weeks since as yet uncharacterized self-DNA, presumably normally digested by Trex1, activates intracellular DNA sensors which triggers cytokine production and causes lethal inflammatory aggravated myocarditis. Recent data indicates that loss of STING can extend the lifespan of Trex1^{-/-} mice although the causes are unknown. These studies were extended and it was noted that there were slightly elevated levels of type I IFN production in Trex1 deficient BMDC (Trex1^{-/-} BMDC) exposed to dsDNA90. Significantly, loss of STING (Sting^{-/-} Trex1^{-/-} BMDC) eliminated the ability of DNA to augment type I IFN production in BMDM's deficient in Trex1. Interestingly, a size reduction of the hearts of Sting^{-/-} Trex1^{-/-}, Sting^{-/+} Trex1^{-/-} was observed when compared to Trex1^{-/-} mice. Evidence of myocarditis was also note to be dramatically reduced in Sting^{-/-} Trex1^{-/-} compared to Trex1^{-/-} alone. In addition, anti-nuclear autoantibody (ANA) observed to be highly prevalent in the sera of Trex1^{-/-} mice, was almost completely absent in the sera of Sting^{-/-} Trex1^{-/-} mice. Microarray analysis demonstrated dramatically, reduced levels of pro-inflammatory genes in the hearts of Sting^{-/-} Trex1^{-/-}, Sting^{-/+} Trex1^{-/-} compared to Trex1^{-/-} mice. Collectively, these data indicates that STING is responsible for pro-inflammatory gene induction in Trex1 deficient mice and plausibly AGS.

### Example 4

### Screening of Kinases for S366 of STING

The activity of 217 protein kinase targets were evaluated against 2 peptides (A366 and S366) as substrate. Protein kinases were mixed with each peptide and ³³P-ATP and then activity (CPM) was measured. The below kinases were identified as phosphorylating S366 in STING. Identification of kinases which target this serine opens up avenues for drug discovery. Drugs that target this association may inhibit STING activity and be used for therapeutic purposes to inhibit STING activity. STING over activity may lead to inflammatory diseases which can exacerbate cancer.

TABLE 4
Screening of kinases for S366 of STING

| | Activity (cpm) | | |
|---|---|---|---|
| Protein Kinase | A366 peptide | S366 peptide | Fold induction |
| CK1δ | 15569 | 25185 | 1.62 |
| GRK7 | 7263 | 13124 | 1.81 |
| IKKα | 2432 | 6889 | 2.83 |
| IRAK4 | 7435 | 50353 | 6.77 |
| MEKK1 | 11316 | 24866 | 2.20 |
| NEK2 | 8702 | 162331 | 18.65 |
| NEK6 | 5512 | 35556 | 6.45 |
| NEK7 | 3619 | 19112 | 5.28 |
| NEK9 | 6977 | 26284 | 3.77 |
| PIM2 | 1414 | 3715 | 2.63 |
| PKCι | 6812 | 12820 | 1.88 |
| RIPK5 | 1327 | 15966 | 12.03 |
| TBK1 | 6603 | 16651 | 2.52 |
| ULK1 | 12221 | 209014 | 17.10 |
| ULK2 | 7377 | 155880 | 21.13 |

### Example 5

### STING is Responsible for Inflammation-Associated Cancer

STING WT and STING^{-/-} animals were treated with DNA damaging agents and mice lacking STING were resistant to tumor formation. This is because infiltrating immune cells such as dendritic cells, macrophages etc eat the damaged cells that have undergone necrosis or apoptosis and the DNA or other ligands from such cells activate STING and the production of cytokines that promote tumor formation. STING may be involved in facilitating tumor progression in a wide variety of other cancers.

FIGS. 29A-D show that STING deficient mice are resistant to DMBA induced inflammation and skin oncogenesis: STING^{+/+} and STING^{-/-} mice were either mocked treated with acetone or treated with 10 μg of DMBA on the shaved dorsal weekly for 20 weeks. FIG. 29A: STING deficient animals are resistant to DNA-damaging agents that cause skin cancer. Percentages of skin tumor-free mice were shown in the Kaplan-Meier curve. FIG. 29B: Pictures of representative mice of each treatment groups were shown. FIG. 29C: Histopathological examinations were performed by H&E staining on mock or DMBA treated skin/skin tumor biopsies. Images were taken at 20× magnification. FIG. 29D: Cytokine upregulation in STING expressing mice exposed to carcinogens. RNAs extracted from mock or DMBA treated skin/skin tumor biopsies were analyzed by Illumina Sentrix BeadChip Array (Mouse WG6 version 2) in duplicate. Total gene expression was analyzed. Most variable genes were selected. Rows represent individual genes; columns represent individual samples. Pseudo-colors indicate transcript levels below, equal to, or above the mean. Gene expression; fold change log 10 scale ranges between −5 to 5. No cytokines were observed in the skin of STING-deficient animals.

What is claimed is:

1. A method for modulating an immune response in a subject having a disease or disorder associated with aberrant STING function, the method comprising the step of administering to the subject an amount of a pharmaceutical composition comprising an agent which modulates STING function and a pharmaceutically acceptable carrier, wherein amount the pharmaceutical composition is effective to ameliorate the aberrant STING function in the subject.

2. The method of claim 1, wherein the agent is a small molecule that increases STING function.

3. The method of claim 1, wherein the agent is a small molecule that decreases STING function.

4. The method of claim 1, wherein the agent is a nucleic acid molecule that binds to STING under intracellular conditions.

5. The method of claim 5, wherein the nucleic acid molecule is a single-stranded DNA between 40 and 150 base pairs in length.

6. The method of claim 5, wherein the nucleic acid molecule is a double-stranded DNA between 40 and 150 base pairs in length.

7. The method of claim 5, wherein the nucleic acid molecule is a double-stranded DNA between 60 and 120 base pairs in length.

8. The method of claim 5, wherein the nucleic acid molecule is a double-stranded DNA between 80 and 100 base pairs in length.

9. The method of claim 5, wherein the nucleic acid molecule is a double-stranded DNA between 85 and 95 base pairs in length.

10. The method of claim 4, wherein the nucleic acid molecule comprises nuclease-resistant nucleotides.

11. The method of claim 4, wherein the nucleic acid molecule is associated with a molecule that facilitates transmembrane transport of the nucleic acid molecule.

12. The method of claim 1, wherein the disease or disorder is a DNA-dependent inflammatory disease.

13. A method of treating cancer in a subject having a cancerous tumor infiltrated with inflammatory immune cells, the method comprising the step of administering to the subject an amount of a pharmaceutical composition comprising an agent which downregulates STING function or expression and a pharmaceutically acceptable carrier, wherein amount the pharmaceutical composition is effective to reduce the number of inflammatory immune cells infiltrating the cancerous tumor by at least 50%.
